SEQUENCE LISTING (75 sequences)

SEQ ID NO: 1 - protein, 8 aa, Artificial Sequence
  model peptide from mouse ovalbumin
SIINFEKL

SEQ ID NO: 2 - protein, 19 aa, Artificial Sequence
  frameshifted peptide
LSLEEGAQSQLTTAMDYLR

SEQ ID NO: 3 - protein, 14 aa, Artificial Sequence
  frameshifted peptide
GPSSSPGGANPVMK

SEQ ID NO: 4 - protein, 17 aa, Artificial Sequence
  frameshifted peptide
LGCVLSGDTPGLLPIPR

SEQ ID NO: 5 - protein, 16 aa, Artificial Sequence
  frameshifted peptide
ASLMTGKTTWQSSTFL

SEQ ID NO: 6 - protein, 27 aa, Artificial Sequence
  frameshifted peptide
ATGLAMAATTTRPMAITATAPATTTVR

SEQ ID NO: 7 - protein, 25 aa, Artificial Sequence
  frameshifted peptide
IIEPSPTTRPSASLSDPSLLPKIPR

SEQ ID NO: 8 - protein, 22 aa, Artificial Sequence
  frameshifted peptide
AALRPAGALPPLPAHLSVPAAR

SEQ ID NO: 9 - protein, 13 aa, Artificial Sequence
  frameshifted peptide
SGHAQAGALSDCG

SEQ ID NO: 10 - protein, 19 aa, Artificial Sequence
  frameshifted peptide
RTSCSVQTAIPTSTHPSAR

SEQ ID NO: 11 - protein, 21 aa, Artificial Sequence
  frameshifted peptide
RVPTCMTTACCCPAELTSSEG

SEQ ID NO: 12 - protein, 20 aa, Artificial Sequence
  frameshifted peptide
ASHCIALEAKWSTCCLYYSR

SEQ ID NO: 13 - protein, 15 aa, Artificial Sequence
  frameshifted peptide
CLSVTSSPRGTSASP

SEQ ID NO: 14 - protein, 13 aa, Artificial Sequence
  frameshifted peptide
LAAPGHQGCGCDR

SEQ ID NO: 15 - protein, 14 aa, Artificial Sequence
  frameshifted peptide
LQHSEAEISELYSS

SEQ ID NO: 16 - protein, 10 aa, Artificial Sequence
  frameshifted peptide
TLPLKLLTTK

SEQ ID NO: 17 - protein, 15 aa, Artificial Sequence
  frameshifted peptide
AAATSAAPTSETSIR

SEQ ID NO: 18 - protein, 28 aa, Artificial Sequence
  frameshifted peptide
GPGGAPSAADRGPPEPQGQQREDDSDYV

SEQ ID NO: 19 - protein, 15 aa, Artificial Sequence
  frameshifted peptide
SPLSTGDRCSPSPVK

SEQ ID NO: 20 - protein, 31 aa, Artificial Sequence
  frameshifted peptide
LAALAQGAAGPGAHLPDGAAAARPGAGAHAR

SEQ ID NO: 21 - protein, 17 aa, Artificial Sequence
  frameshifted peptide
VLRLQHSEAEISELYSS

SEQ ID NO: 22 - protein, 17 aa, Artificial Sequence
  frameshifted peptide
GSVEGESALEALRPAVK

SEQ ID NO: 23 - protein, 18 aa, Artificial Sequence
  frameshifted peptide
QAQGAGELQGGDQGILPR

SEQ ID NO: 24 - protein, 33 aa, Artificial Sequence
  frameshifted peptide
RGQPGGSEGASRLPELSEGEGPCAGSNSQEPEG

SEQ ID NO: 25 - protein, 26 aa, Artificial Sequence
  frameshifted peptide
LGVHPLSCHGGSLGNMEIPVCLYGVE

SEQ ID NO: 26 - protein, 22 aa, Artificial Sequence
  frameshifted peptide
TVGTADHHGPMTTSSSGGLPTR

SEQ ID NO: 27 - protein, 17 aa, Artificial Sequence
  frameshifted peptide
LIALSIDSVEDHLAGAR

SEQ ID NO: 28 - protein, 24 aa, Artificial Sequence
  frameshifted peptide
AARGPGGAPSAADRGPPEPQGQQR

SEQ ID NO: 29 - protein, 22 aa, Artificial Sequence
  frameshifted peptide
EFRPEDQPGCSGSTANQAGSSR

SEQ ID NO: 30 - protein, 16 aa, Artificial Sequence
  frameshifted peptide
ASCGPGPSGCGALIPK

SEQ ID NO: 31 - protein, 19 aa, Artificial Sequence
  frameshifted peptide
RQAQGAGELQGGDQGILPR

SEQ ID NO: 32 - protein, 29 aa, Artificial Sequence
  frameshifted peptide
NLQEAEEGTNPSLLTSLRLPTGTMTPCAR

SEQ ID NO: 33 - protein, 13 aa, Artificial Sequence
  frameshifted peptide
SGSSSTSGPTQQR

SEQ ID NO: 34 - protein, 16 aa, Artificial Sequence
  frameshifted peptide
AKEESSGCAGCPKVLR

SEQ ID NO: 35 - protein, 23 aa, Artificial Sequence
  frameshifted peptide
EGHGGGHPAAAPPGGAQGAASDH

SEQ ID NO: 36 - protein, 36 aa, Artificial Sequence
  frameshifted peptide
LGQLPGAEGQAEAGAPSLGAGAQCQAQPSGTHHCRE

SEQ ID NO: 37 - protein, 18 aa, Artificial Sequence
  frameshifted peptide
HNEVWMVDFYSPGVILAK

SEQ ID NO: 38 - protein, 19 aa, Artificial Sequence
  frameshifted peptide
VGRPMGMVKTDSGGPSVLK

SEQ ID NO: 39 - protein, 16 aa, Artificial Sequence
  frameshifted peptide
AAGQAQGTALLQHEDR

SEQ ID NO: 40 - protein, 11 aa, Artificial Sequence
  frameshifted peptide
EGGKIEMENLK

SEQ ID NO: 41 - protein, 14 aa, Artificial Sequence
  frameshifted peptide
TLTAMSVVGFPGGR

SEQ ID NO: 42 - protein, 14 aa, Artificial Sequence
  frameshifted peptide
ITTSHTEAAAGATN

SEQ ID NO: 43 - protein, 13 aa, Artificial Sequence
  REV 1 peptide
MISSTSSEMQQTK

SEQ ID NO: 44 - protein, 17 aa, Artificial Sequence
  REV 2 peptide
NCNEVVWQPGMLTAAII

SEQ ID NO: 45 - protein, 17 aa, Artificial Sequence
  REV 3 peptide
QEDLFEQLETLHSINEI

SEQ ID NO: 46 - protein, 10 aa, Artificial Sequence
  frameshifted peptide
VPLSLAEHAL

SEQ ID NO: 47 - protein, 9 aa, Artificial Sequence
  frameshifted peptide
SPLSSLSTL

SEQ ID NO: 48 - protein, 9 aa, Artificial Sequence
  frameshifted peptide
QFLAEILQV

SEQ ID NO: 49 - protein, 9 aa, Artificial Sequence
  frameshifted peptide
SPTLSQCSL

SEQ ID NO: 50 - protein, 10 aa, Artificial Sequence
  frameshifted peptide
MVSPLAGVPK

SEQ ID NO: 51 - protein, 9 aa, Artificial Sequence
  frameshifted peptide
VGQEGLVSL

SEQ ID NO: 52 - protein, 9 aa, Artificial Sequence
  frameshifted peptide
SPALRTLTL

SEQ ID NO: 53 - protein, 9 aa, Artificial Sequence
  frameshifted peptide
APSGVAAGL

SEQ ID NO: 54 - protein, 9 aa, Artificial Sequence
  frameshifted peptide
LFLSHLEEI

SEQ ID NO: 55 - protein, 9 aa, Artificial Sequence
  frameshifted peptide
VAAAESHPL

SEQ ID NO: 56 - protein, 9 aa, Artificial Sequence
  frameshifted peptide
TSLVNLSTL

SEQ ID NO: 57 - protein, 9 aa, Artificial Sequence
  frameshifted peptide
SPRGPPSSL

SEQ ID NO: 58 - protein, 9 aa, Artificial Sequence
  RFX1-based peptide
TLVEDLLEV

SEQ ID NO: 59 - protein, 9 aa, Artificial Sequence
  ILRUN-based peptide
LLTHGLLLL

SEQ ID NO: 60 - protein, 9 aa, Artificial Sequence
  ETF1-based peptide
LLEGLLTTI

SEQ ID NO: 61 - protein, 9 aa, Artificial Sequence
  RHOBTB3-based peptide
TVIGTLLEI

SEQ ID NO: 62 - protein, 11 aa, Artificial Sequence
  SRSF3-based peptide
GLLETHPALLL

SEQ ID NO: 63 - protein, 15 aa, Artificial Sequence
  CNIH4-based peptide
LMSLHLVHLPSQLTC

SEQ ID NO: 64 - protein, 10 aa, Artificial Sequence
  c-myc domain
EQKLISEEDL

SEQ ID NO: 65 - protein, 9 aa, Artificial Sequence
  hemagglutinin tag
YPYDVPDYA

SEQ ID NO: 66 - protein, 4 aa, Artificial Sequence
  tryptophan-containing region
  misc_feature (2)..(3): Xaa can be any naturally occurring amino acid
WXXW

SEQ ID NO: 67 - protein, 5 aa, Artificial Sequence
  tryptophan-containing region
  misc_feature (2)..(4): Xaa can be any naturally occurring amino acid
WXXXW

SEQ ID NO: 68 - protein, 6 aa, Artificial Sequence
  tryptophan-containing region
  misc_feature (2)..(5): Xaa can be any naturally occurring amino acid
WXXXXW

SEQ ID NO: 69 - protein, 7 aa, Artificial Sequence
  tryptophan-containing region
  misc_feature (2)..(6): Xaa can be any naturally occurring amino acid
WXXXXXW

SEQ ID NO: 70 - protein, 8 aa, Artificial Sequence
  tryptophan-containing region
  misc_feature (2)..(7): Xaa can be any naturally occurring amino acid
WXXXXXXW

SEQ ID NO: 71 - protein, 9 aa, Artificial Sequence
  tryptophan-containing region
  misc_feature (2)..(8): Xaa can be any naturally occurring amino acid
WXXXXXXXW

SEQ ID NO: 72 - protein, 10 aa, Artificial Sequence
  tryptophan-containing region
  misc_feature (2)..(9): Xaa can be any naturally occurring amino acid
WXXXXXXXXW

SEQ ID NO: 73 - protein, 11 aa, Artificial Sequence
  tryptophan-containing region
  misc_feature (2)..(10): Xaa can be any naturally occurring amino acid
WXXXXXXXXXW

SEQ ID NO: 74 - RNA, 28 nt, Artificial Sequence
  hypothetical RNA template
  misc_feature (1)..(13): n is a, c, g, or u
  misc_feature (17)..(28): n is a, c, g, or u
nnnnnnnnnnnnnuggnnnnnnnnnnnn

SEQ ID NO: 75 - RNA, 28 nt, Artificial Sequence
  hypothetical RNA template
  misc_feature (4)..(28): n is a, c, g, or u
uggnnnnnnnnnnnnnnnnnnnnnnnnn

(19) United States
(12) Patent Application Publication
(10) Pub. No.: US 2023/0390371 A1
(43) Pub. Date: Dec. 7, 2023

(54) ANTI-TUMOR IMMUNITY INDUCES THE PRESENTATION OF ABERRANT PEPTIDES

(71) Applicant: STICHTING HET NEDERLANDS KANKER INSTITUUT-ANTONI VAN LEEUWENHOEK ZIEKENHUIS, Amsterdam (NL)

(72) Inventors: Reuven AGAMI, Amsterdam (NL); Abhijeet PATASKAR, Amsterdam (NL); Julien CHAMPAGNE, Amsterdam (NL); Yardena SAMUELS, Rehovot (IL); Osnat BARTOK, Rehovot (IL)

(21) Appl. No.: 18/032,183

(22) PCT Filed: Oct. 18, 2021
(86) PCT No.: PCT/NL2021/050629

§ 371 Date: Apr. 16, 2023

(30) Foreign Application Priority Data
Oct. 16, 2020 (EP) 20202435.2
Dec. 16, 2020 (EP) 20214612.2
Apr. 1, 2021 (EP) 21166757.1

(51) Int. Cl. A61K 39/00; C12P 21/02; C07K 14/435

(52) U.S. Cl. CPC A61K 39/00119; C12P 21/02; C07K 14/435; A61K 2039/70; A61K 2039/53; A61K 2039/876; A61K 2039/55522

(57) ABSTRACT

The invention relates to methods of producing at least one out-of-frame peptide of 5-40 amino acid residues by a cell, and to methods of identifying said at least one out-of-frame peptide. The invention further relates to the identified out-of-frame peptides, and epitopes and polyepitopes encompassing at least one of said out-of-frame peptides, and to a nucleic acid molecule encoding at least one of said out-of-frame peptides. The invention further relates to methods of inducing an immune response against at least one out-of-frame peptide, method of treating an individual suffering from a tumor, comprising providing said individual with at least one out-of-frame peptide, and to a pharmaceutical composition, comprising at least one out-of-frame peptide. The invention additionally relates to a kit for detecting expression of at least one out-of-frame peptide, and to methods of typing an individual for presence or absence of a cell that expresses at least one out-of-frame peptide.

FIELD: The invention generally relates to immune stimulatory compositions comprising at least one out-of-frame peptide of 5-40 amino acid residues that is produced by a cell upon tryptophan deprivation.

### INTRODUCTION

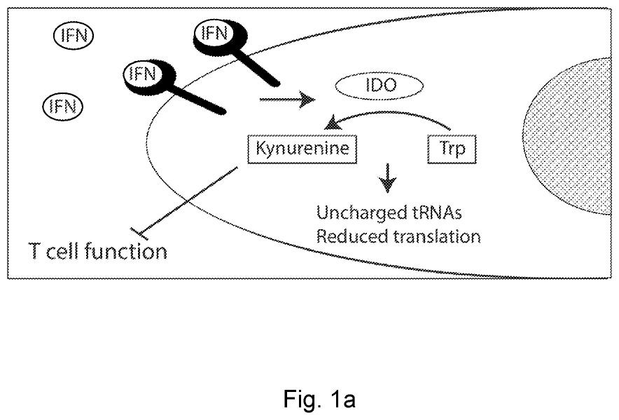

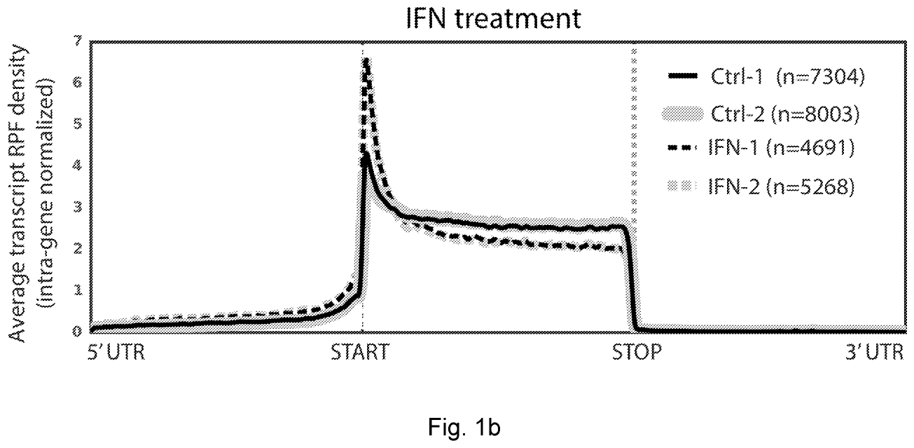

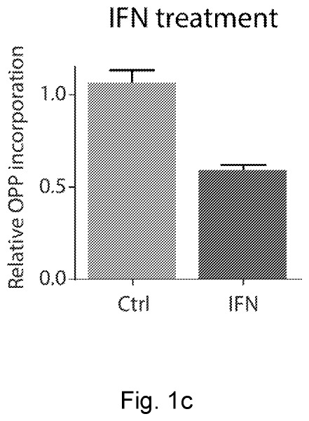

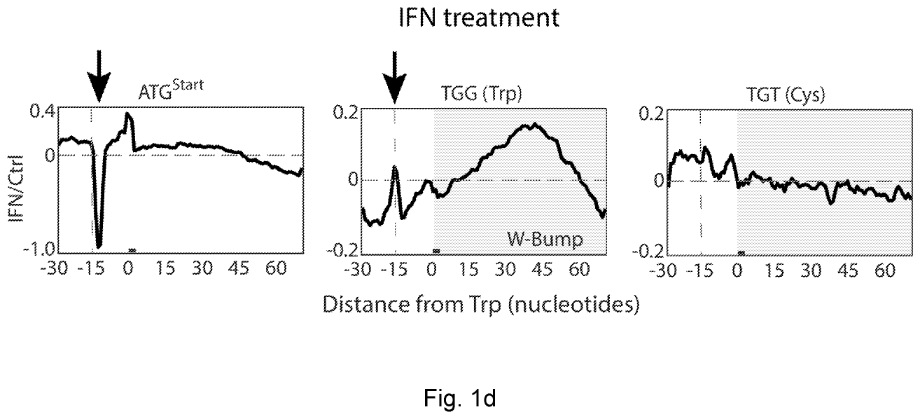

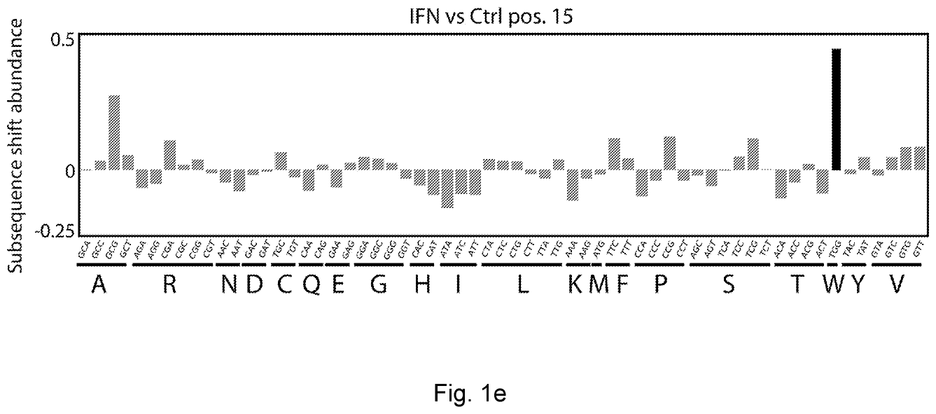

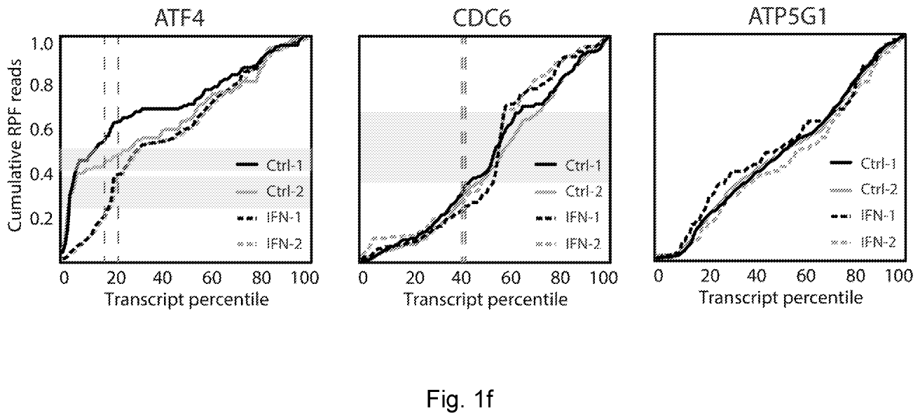

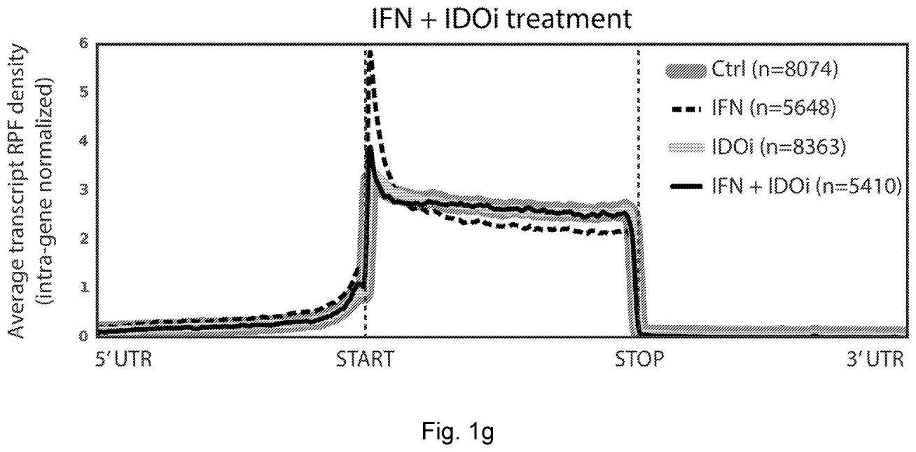

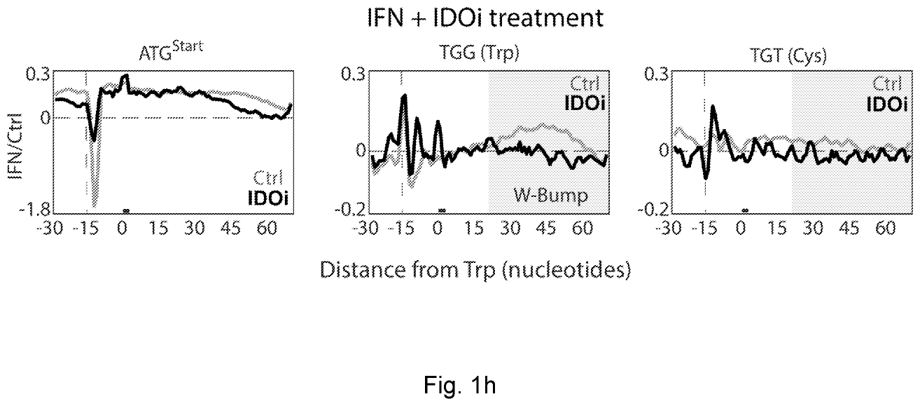

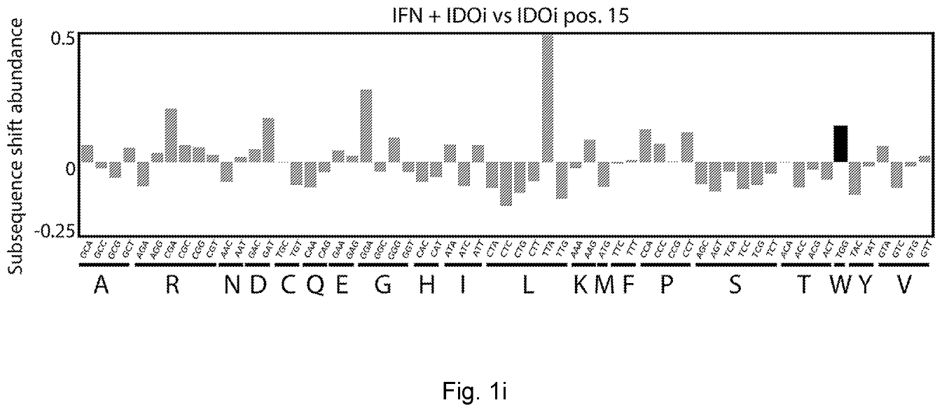

IFNγ-induced indoleamine 2,3-dioxygenase 1 (IDO1)-mediated tryptophan (Trp) deprivation stimulates the uncharged tRNA sensing machinery pathway, whose main components are EIF2AK4 (general control nonderepressible protein 2, GCN2), eIF2α, and the activating transcription factor 4 (ATF4)^{4,13-17}. Furthermore, GCN2 can also be induced by ribosome stalling, independent of tRNA charging, leading to inhibition of translation elongation¹⁸, and amino acid deprivation can activate ZAKα, a MAPKKK enzyme whose activation induces a cellular stress response and apoptosis¹⁹. When active, the EIF2AK4-ATF4 cascade suppresses initiation of protein synthesis (FIG. 1a). Conversely, cancers compensate for IFNγ-induced tryptophan deprivation both by upregulating the expression of several amino acid transporters, which enhance tryptophan and glutamine uptake, and WARS, the tryptophanyl tRNA synthetase. This enables cancer cells to survive and quickly recover once tryptophan is replenished^{5,20}. However, the long-term impact of sustained IFNγ-mediated tryptophan depletion on melanoma cells, remains largely unknown.

### BRIEF DESCRIPTION OF THE INVENTION

The invention provides a method of producing at least one out-of-frame peptide of 5-40 amino acid residues by a cell, said method comprising incubating said cell in a growth medium, reducing the amount of tryptophan in said cell, thus producing an out-of-frame peptide of 5-40 amino acid residues by the cell. The amount of tryptophan is preferably reduced in said cell by providing growth medium that is depleted of tryptophan, by incubation of the cells in the presence of interferon gamma, by activation of indoleamine 2,3-dioxygenase 1 (IDO1), indoleamine 2, 3-dioxygenase 2 (IDO2), and/or tryptophan 2, 3-dioxygenase (TDO) in the cells, or by a combination thereof. Some of the cells present 8-22 amino acid residues of the out-of-frame peptide on the surface of said cell by MHC, preferably by MHC class I. Said cell especially is a tumor cell such as a melanoma cell.

The invention further provides a method of identifying at least one out-of-frame peptide of 5-40 amino acid residues, said method comprising providing a cell in which the amount of tryptophan has been reduced, and identifying at least one out-of-frame peptide of 5-40 amino acid residues that is produced by said cell, preferably identifying a peptide of 8-22 amino acid residues that is presented by MHC on the surface of said cell.

The invention further provides an out-of-frame peptide of 5-40 amino acid residues that is produced by a cell upon reduction of tryptophan in said cell. Said out-of-frame peptide preferably is selected from Table 1. The invention thus provides a peptide of 5-40 amino acid residues that is produced by a cell upon reduction of tryptophan in said cell. Said peptide preferably is a peptide of 8-22 amino acid residues that is presented by MHC on the surface of said cell. Said peptide preferably is selected from Table 1.

The invention further provides a T cell epitope comprising 8-22 amino acid residues, more preferred 8-13 amino acid residues, comprising at least part of an out-of-frame peptide of 5-40 amino acid residues that is produced by a cell upon reduction of tryptophan in said cell. Said out-of-frame peptide preferably is selected from Table 1. The invention thus provides a T cell epitope comprising 8-22 amino acid residues, more preferred 8-13 amino acid residues, comprising at least part of a peptide of 5-40 amino acid residues that is produced by a cell upon reduction of tryptophan in said cell. Said T cell epitope preferably comprises one or more peptides with SEQ ID NOs 46-63, more preferably SEQ ID NOs 46-57 or SEQ ID NOs 58-63.

The invention further provides a polyepitope, comprising 2-50, preferably 5-individual T cell epitopes according to claim 7, preferably each contained within a sequence of 8-40 amino acid residues, which individual epitopes may be alternated by spacer sequences, preferably of 1-10 amino acid residues.

The invention further provides a B cell epitope, comprising at least one out-of-frame peptide of 5-40 amino acid residues according to claim 6.

The invention further provides a nucleic acid molecule, encoding the T cell epitope of claim 7, the polyepitope of claim 8, the B cell epitope of claim 9, said nucleic acid molecule preferably being a RNA molecule, or a DNA molecule that expresses said polyepitope upon delivery to a suitable cell.

The invention further provides a T cell, comprising a T cell Receptor TCR) that is directed against the T cell epitope of the invention.

The invention further provides a method of inducing an immune response in an individual against at least one out-of-frame peptide of 5-40 amino acid residues that is produced by a cell, said method comprising providing said individual with the T cell epitope of claim 7, the polyepitope of claim 8, the B cell epitope of claim 9, the nucleic acid molecule of claim 10, or a combination thereof.

The invention further provides a method of inducing an immune response in an individual against at least one out-of-frame peptide of 5-40 amino acid residues that is produced by a cell, said method comprising providing said individual with a T cell epitope of the invention, a polyepitope of the invention, the B cell epitope of the invention, a nucleic acid molecule of the invention, a T cell of the invention, or a combination thereof.

The invention further provides a method of treating an individual suffering from a tumor such as a melanoma, comprising providing said individual with the T cell epitope of claim 7, the polyepitope of claim 8, the B cell epitope of claim 9, the nucleic acid molecule of claim 10, or a combination thereof. A preferred combination encompasses providing said individual with the nucleic acid molecule of claim 10, preferably a mRNA molecule, and the T cell of claim 11. Said individual may comprise a cell such as a tumor cell that expresses the at least one out-of-frame peptide of 5-40 amino acid residues. Said individual preferably is further provided with interferon gamma, optionally combined with a tryptophan-low or tryptophan-free diet, an immune checkpoint inhibitor, or both, whereby said interferon gamma and/or immune checkpoint inhibitor may be administered prior to, simultaneously with, or following administration of the T cell epitope of claim 7, the polyepitope of claim 8, the B cell epitope of claim 9, the nucleic acid molecule of claim 10, or a combination thereof. Said individual preferably is additionally provided with an inducer of kynureninase. such as an kynureninase-expressing construct.

The invention further provides a method of treating an individual suffering from a tumor such as a melanoma, comprising providing said individual with the T cell epitope of the invention, the polyepitope of the invention, the B cell epitope of the invention, the nucleic acid molecule of the invention, the T cell of the invention, or a combination thereof. Said individual preferably comprises a cell such as a tumor cell that expresses the at least one out-of-frame peptide of 5-40 amino acid residues according to the invention. Said individual preferably is further provided with interferon gamma, optionally combined with a tryptophan-low or tryptophan-free diet, an immune checkpoint inhibitor, or both, whereby said interferon gamma and/or immune checkpoint inhibitor may be administered prior to, simultaneously with, or following administration of the T cell epitope of the invention, the polyepitope of the invention, the B cell epitope of the invention, the nucleic acid molecule of the invention, the T cell of the invention, or a combination thereof. Said individual preferably is additionally provided with an inducer of kynureninase. such as an kynureninase-expressing construct.

The invention further provides a pharmaceutical composition, comprising the T cell epitope of claim 7, the polyepitope of claim 8, the B cell epitope of claim 9, the nucleic acid molecule of claim 10, or a combination thereof and, optionally, an accessory molecule such as an adjuvant, an immune checkpoint inhibitor, an immune stimulating molecule such as a chemokine and/or a cytokine, or a combination thereof.

The invention further provides a pharmaceutical composition, comprising the T cell epitope of the invention, the polyepitope of the invention, the B cell epitope of the invention, the nucleic acid molecule of the invention, the T cell of the invention, or a combination thereof and, optionally, an accessory molecule such as an adjuvant, an immune checkpoint inhibitor, an immune stimulating molecule such as a chemokine and/or a cytokine, inducer of kynureninase, or a combination thereof.

The invention further provides a kit for detecting expression of at least one out-of-frame peptide of 5-40 amino acid residues by a cell, said kit comprising an antibody, T-cell, or combination thereof, that specifically recognizes said at least one out-of-frame peptide of 5-40 amino acid residues, said kit further comprising means for detection of said antibody, T-cell, or combination thereof.

The invention further provides a method of typing an individual for presence or absence of a cell that expresses at least one out-of-frame peptide of 5-40 amino acid residues, said method comprising incubating a sample comprising cells of the individual with the kit of claim 16, and detecting presence or absence of binding of said antibody, T-cell, or combination thereof, to a cell in said sample.

### FIGURE LEGENDS

FIG. 1: IFNγ induces IDO1-mediated ribosome pausing on tryptophan codons, and form W-Bumps downstream thereof.
(a) A schematic model depicting the effect of IFNγ (IFN) signaling on IDO1 positive cells. IFNγ induction leads to an increase in IDO1 expression, an enzyme catalyzing the conversion of tryptophan to kynurenine. On the one hand, this leads to an increase in uncharged tRNAs which negatively affects protein translation process. On the other hand, the production of kynurenine inhibits T cell function.
(b) Metagene density profiles depicting global shifts of Ribosomal Protected Fragments (RPFs) to the start of the coding sequence in 12T cells upon IFNγ treatment (red and yellow) as compared to control (black and grey). On the y-axis: intra-gene normalized RPF density. (c) Quantification of flow cytometry analysis of OP-Puro incorporation assays as a readout for nascent protein synthesis. 12T cells were either mock treated (Ctrl) or treated with IFNγ for 48 hours. Bars represent averages plus standard deviation of three independent experiments. (d) Diricore analysis line-plots depicting differential ribosome occupancy in 12T cells (5′-RPF) at −30 to +60 codons surrounding the initiator ATG codon (ATGStart, left, in green), tryptophan codon (center, in red) and cysteine codon (right, in grey). Y-axis: The ratio between the number of reads in IFNγ versus control conditions. (e) Diricore analysis bar-plots depicting differential codon usage (at position 15 of the RPFs) in IFNγ versus the control conditions in 12T cells. (f) Diricore line-plots depicting cumulative signal of RPFs across the coding region normalized into percentiles for ATF4 (left), CDC6 (middle) and ATP5G1 (right) genes in 12T control (dark grey, grey) and IFNγ-treated cells (yellow, red). Grey areas mark the sites of W-Bumps as seen by a sharp rise in the number of reads in IFNγ condition downstream of the tryptophan codons (vertical dashed lines). (g) Metagene RPF density profiles for control (black), IFNγ (red), IDOi (grey) and IFNγ+IDOi (green) treated 12T cells. (h) Diricore line-plots comparing IFNγ and IFNγ+IDOi conditions, in 12T cells (i) Diricore bar-plot depicting differential codon usage (at position 15 of the RPFs) comparing IDOi against IFNγ+IDOi treatment of 12T cells.

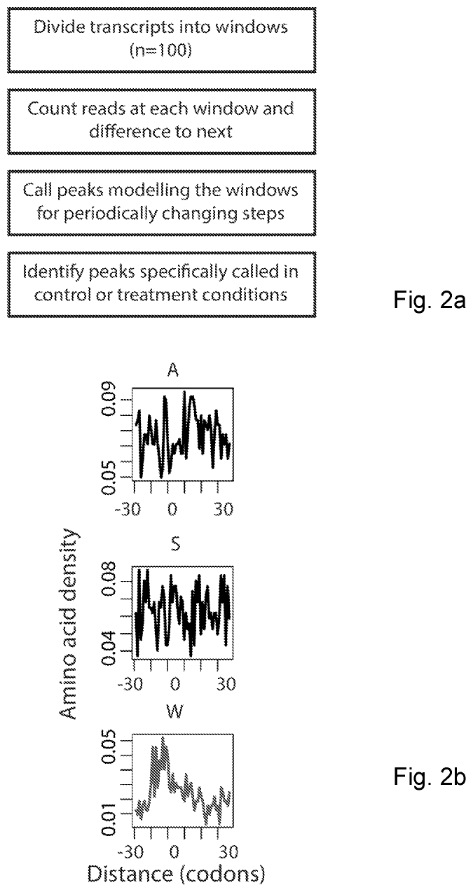

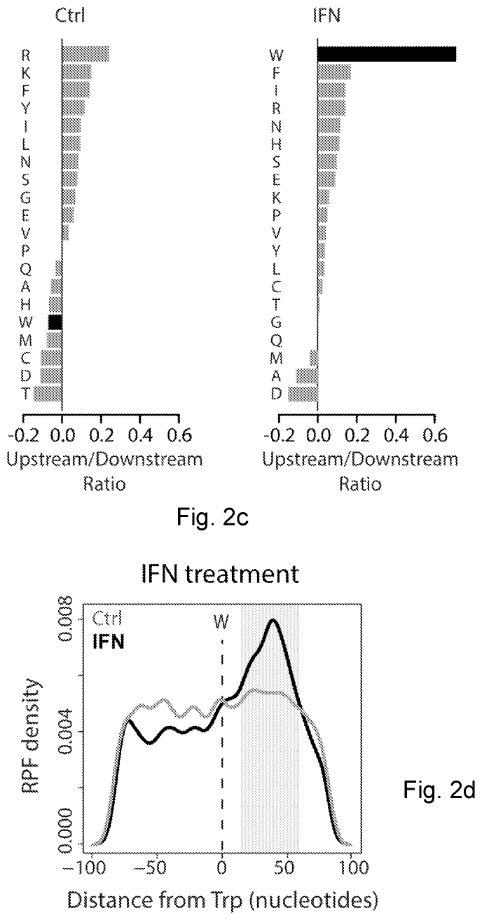

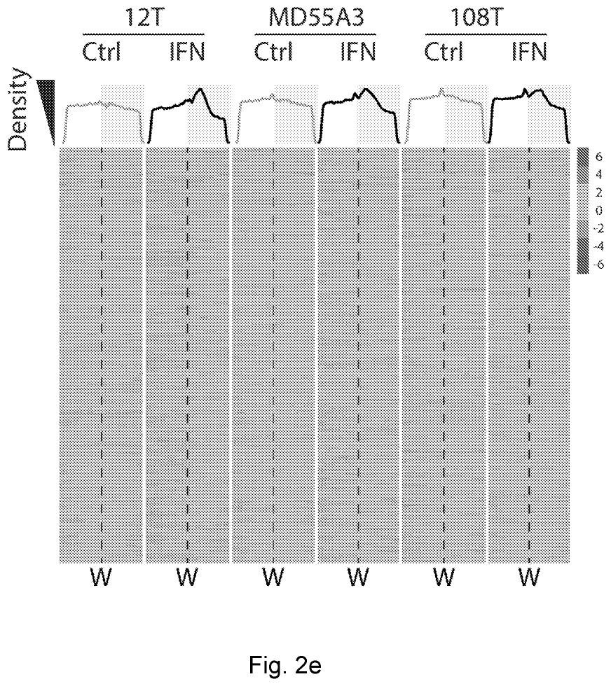

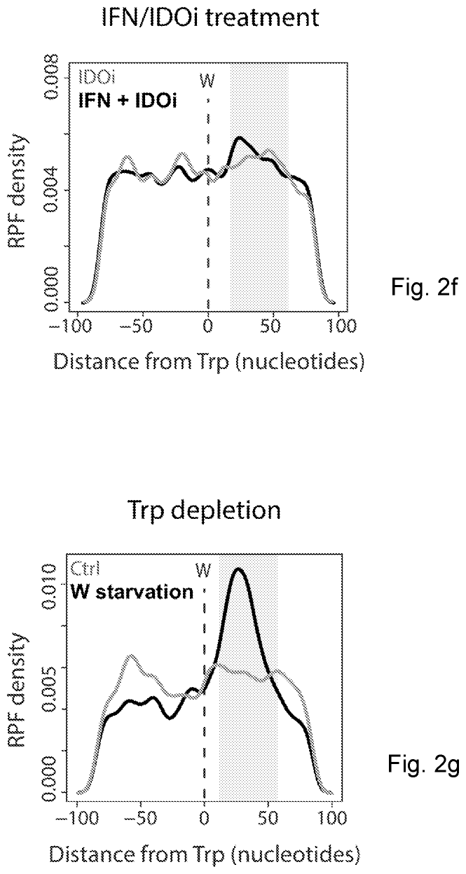

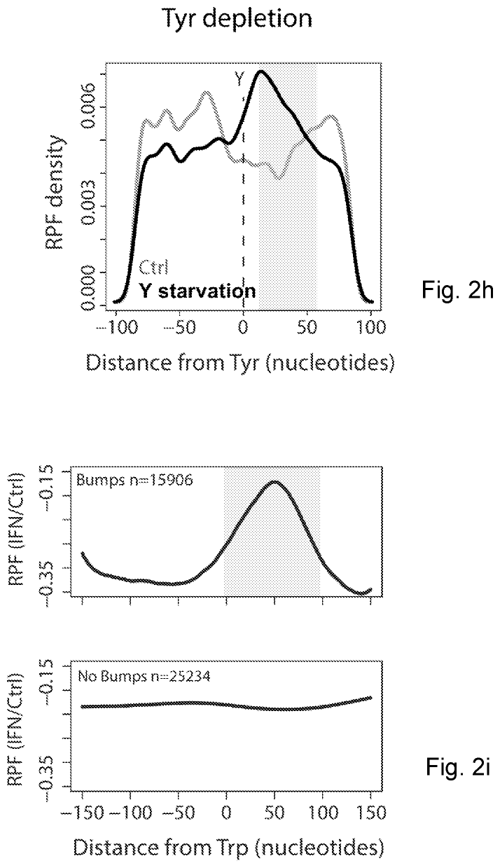

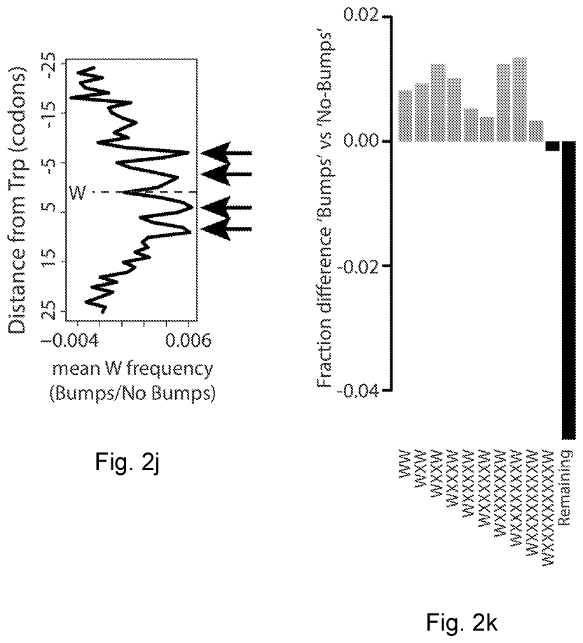

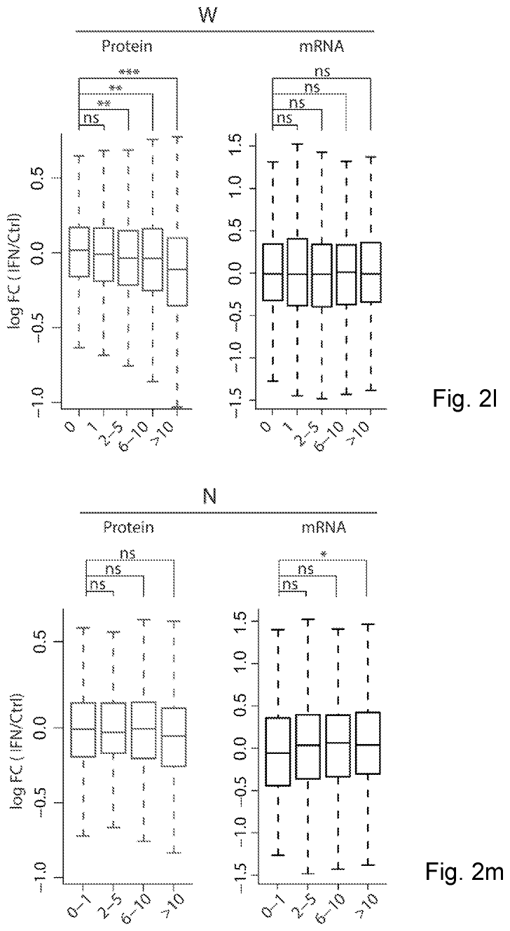

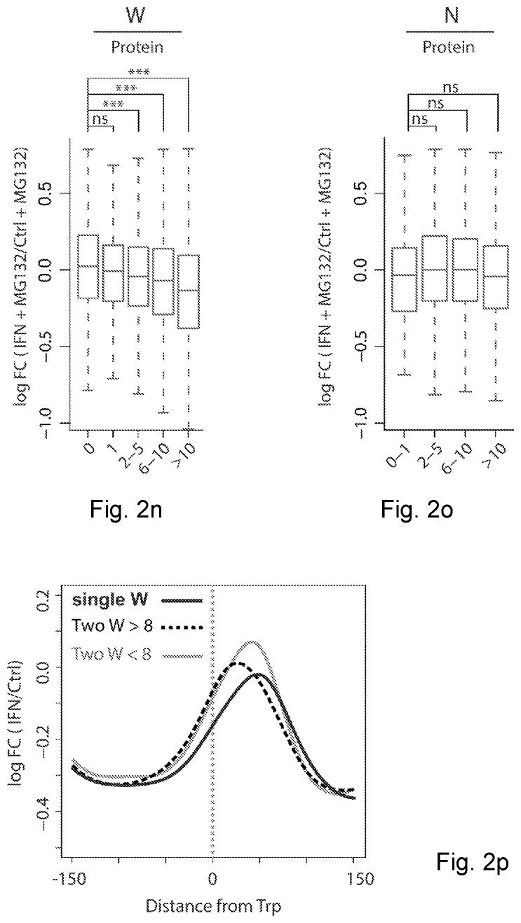

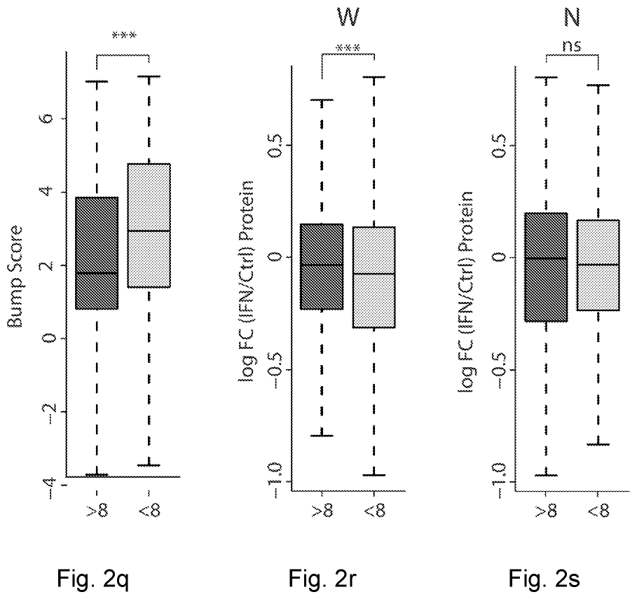

FIG. 2: IFNγ-induced W-Bump formation is associated with the presence of multiple tryptophan codons within a region of eight codons, and is indicative of a reduction in protein synthesis.
(a) Computational approach for unbiased detection of Bumps in comparative ribosome profiling experiments. The algorithm scans transcripts in 100 windows of equal length for peaks in ribosome occupancy and filters for differential peaks in treated versus untreated samples. (b) Density of codons for alanine (top), serine (middle) tryptophan (bottom) in the region of 60 codons upstream and downstream of the peak of bumps identified with bump-finder in ribosome profiling data of IFNγ-treated 12T cells. (c) Ratio between upstream and downstream reads in regions 30 codons from the peak of bumps identified in control (left) and IFNγ-treated conditions (right) for codons for every amino acid. (d) Density of RPFs 200 nucleotides surrounding the tryptophan codon closes to the identified bumps in both control (grey line) and IFNγ-treated (red line) 12T cells (e) Densities (upper lines) and heatmaps (lower panel) of ribosomal P-sites in the area of 100 nucleotides surrounding every tryptophan codon in control and IFNγ conditions in three cell types (12T, MD55A3 and 108T). (f) Density of RPFs 200 nucleotides surrounding the tryptophan codon in IDOi (grey line) and IFNγ+IDOi (red line) treated 12T cells. (g) Density of RPFs 200 nucleotides surrounding the tryptophan codon in control (grey line) and tryptophan depletion (red line) 12T cells. (h) Density of RPFs 200 nucleotides surrounding the tyrosine codon in control (grey line) and tyrosine depleted (red line) 12T cells. (i) Classification of all transcripts containing codons for tryptophan into two sets, one group associated with W-Bumps (‘Bumps’, upper panel) and the other which is not associated with bumps (‘No-bumps’, lower panel). Graphs indicate the RPF density in the region of 300 nucleotides surrounding the tryptophan codon, with the bump area shaded in grey. (j) A line plot depicting mean tryptophan codon enrichment in the Bumps group over the no-Bumps group at 25 codons across the tryptophan codons. Red arrows indicate the enrichment of multiple alternating tryptophan codons within a region of eight codons in the Bumps group in comparison to the No-Bumps group. (k) A bar plot depicting the enrichment of the occurrence of two tryptophan codons within a region of eight codons in the ‘Bumps’ group over the ‘No-Bumps’ group. W indicates a codon for tryptophan, whereas X indicates all other amino acids than tryptophan. (1) Boxplots depicting log fold change in the levels of protein (red; average of three replicates) and mRNA (black; average of two replicates) in IFNγ versus control treated cells. Proteins were grouped according to the number of tryptophans in the protein sequence. Test: Wilcoxon Test; ns=not significant, ** p<0.005 and *** p<0.0005. (m) Boxplots depicting log fold change in the levels of protein (red; average of three replicates) and mRNA (black; average of two replicates) in IFNγ versus control treated cells. Proteins were grouped according to the number of asparagines in the protein sequence. Test: Wilcoxon Test; ns=not significant, *** p<0.05. (n) Boxplots depicting log fold change in the levels of protein (red; average of three replicates) in IFNγ versus control treated cells with inclusion of proteasomal inhibition (MG132 treatment). Proteins were grouped according to the number of tryptophans in the protein sequence. Test: Wilcoxon Test; ns=not significant, *** p<0.0005. (o) Same as 2n, but proteins were grouped according to the number of asparagines in the protein sequence. ns=not significant. (p) Density of RPFs 300 codons across individual tryptophan codons (black line), two tryptophans that are present within a distance of 8 codons (green line) and two tryptophans that are present at a distance greater than 8 codons (red line). (q) A boxplot depicting bump scores for instances of two tryptophan separated by fewer than 8 codons (green) and more than 8 codons (red). Bump scores are calculated in MD55A3 cells. *** p<0.0005. (r) A boxplot depicting protein level changes (log fold change) between IFNγ and control conditions. The graph represents genes which have two tryptophan codons within a distance of 8 codons (green) or genes having a distance of more than 8 codons between two tryptophans (red). Test: Wilcoxon Test; p<0.0005. (s) Same as 2r, but for asparagine, in MD55A3 cells. Test: Wilcoxon Test; ns=not significant.

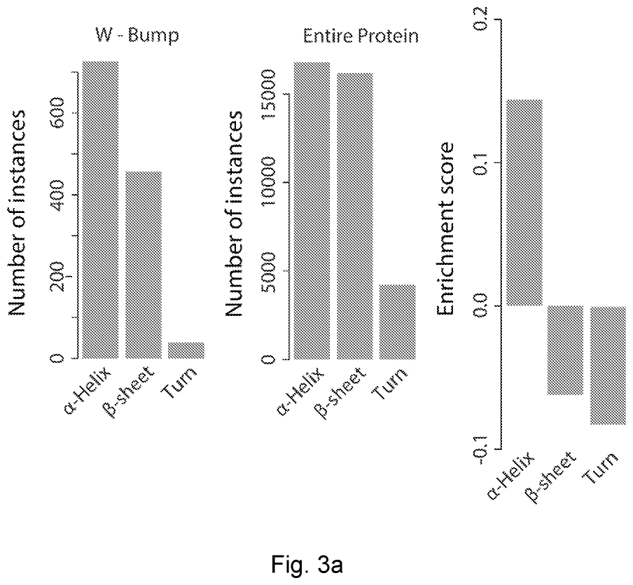

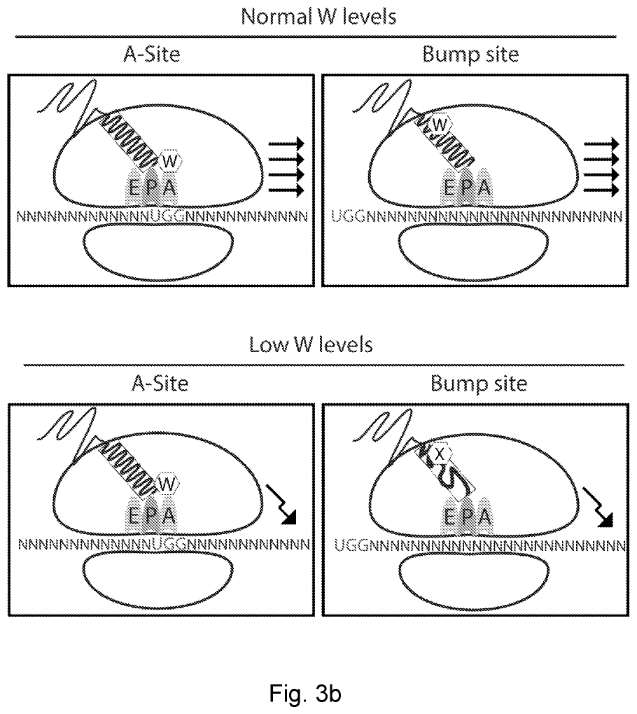

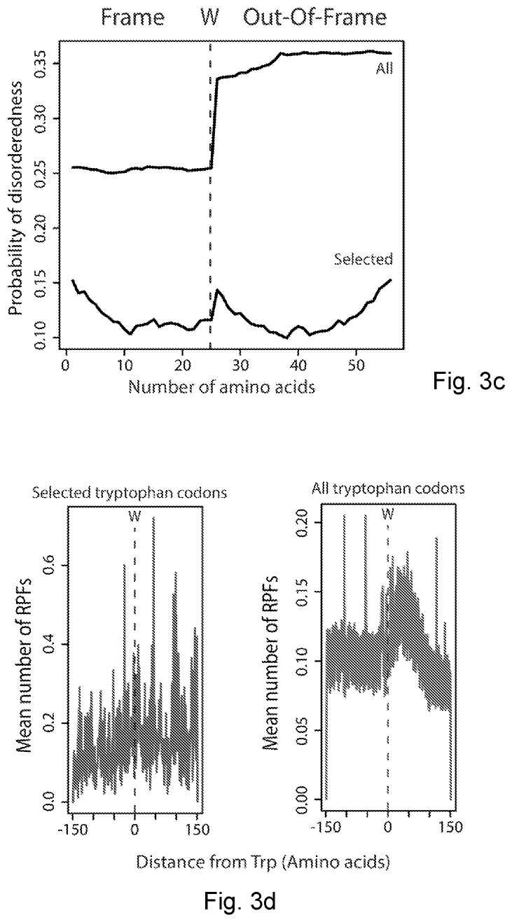

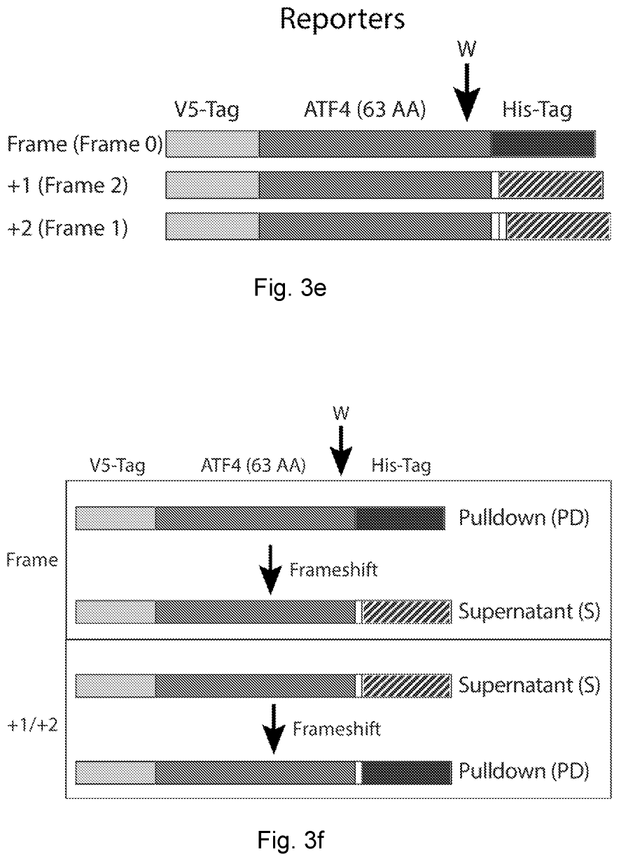

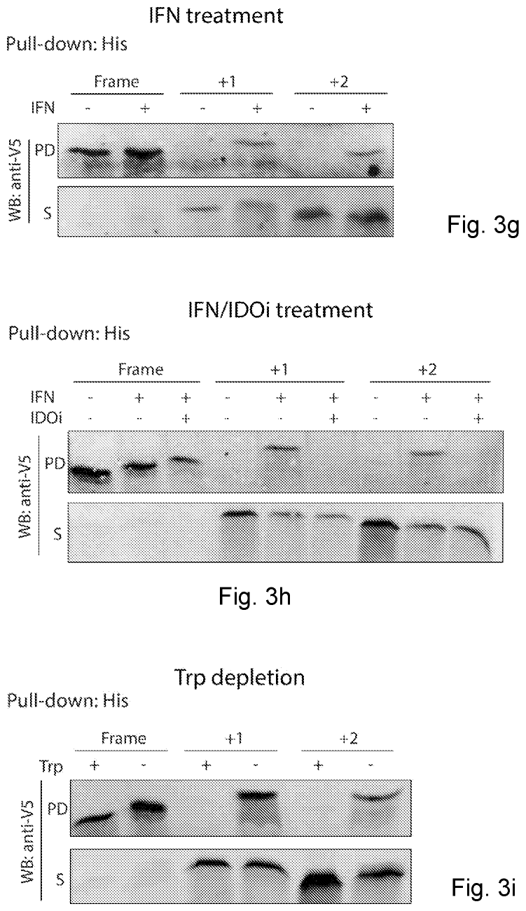

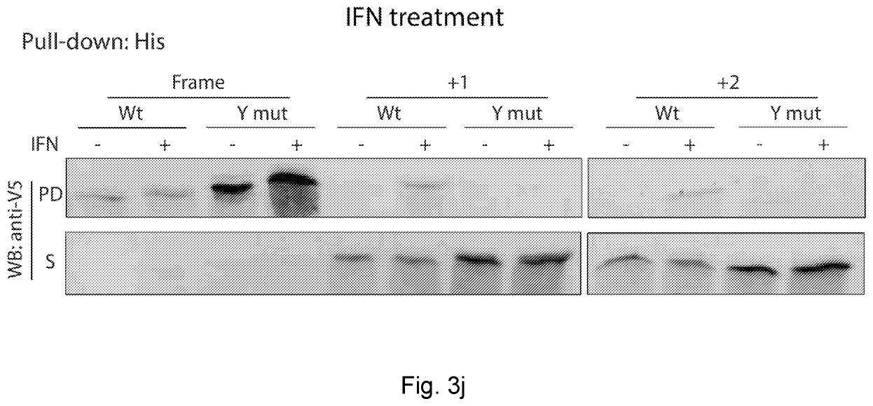

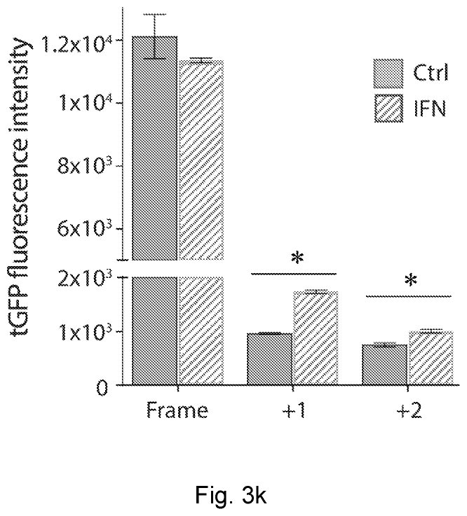

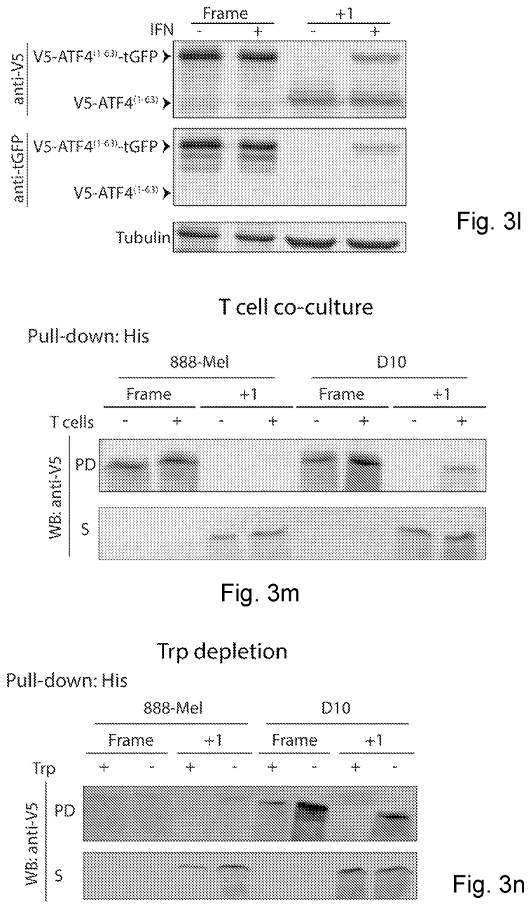

FIG. 3: W-Bumps are associated with disordered out-of-frame peptides, and shortage in tryptophan leads to frameshifting events.
(a) Bar-plots depicting total occurrences of alpha-helices, beta-sheets and turns for tryptophan-containing peptide sequences corresponding to W-Bumps (left) and all remaining peptide sequences (middle), and the difference in percentages (right). (b) A hypothetical model suggesting a possible mechanism causative for W-Bumps. In the normal scenario, ribosomes do not encounter problems when translating a tryptophan and progress translation at regular speed (top panels). Tryptophan shortage, on the other hand, can lead to stalling on the tryptophan codon (bottom left panel), or could in theory induce frameshifting events, leading to aberrant peptide production (lower right panel). Since secondary structure of growing polypeptide chains is attained in the lower tunnel of the ribosome, the loss of an a-helical secondary structure in this tunnel could hamper ribosomal progression. (c) Line-plots depicting the probability of disorderedness of peptide sequences. The position of the tryptophan is indicated by the dashed line. The left half of the graph shows the probability of disorderedness of the in-frame peptide up until the tryptophan, the right part shows the probability for the possible frameshifted (both +1 and −1) peptides downstream of tryptophans. The green line represents the average probability of disorderedness of all peptides (‘All tryptophan codons’, n=36508). A group of outlier candidates that show relatively ordered out-of-frame peptide sequences were selected for comparisons (‘Selected tryptophan codons’, n=94, red). (d) Line-plots depicting mean frequency of RPFs at single nucleotide resolution across all tryptophans (green) and the selected outlier set (red) from panel 3c. (e) A schematic representation of the reporter constructs (V5-ATF4(1-63)-His) used for detection of frameshifting events. The first 63 amino acids of ATF4 (ATF41-63)) were cloned with an N-terminal V5-tag and a C-terminal His-tag. To record frameshifting events, three variants were constructed with the His-tag placed in all three frames. (f) Schematic representation of the final protein sequences that would form due to frameshifting events. The in-frame construct (upper panel) contains a His-tag and would end up in the pulldown (PD) fraction. Whenever a frameshift occurs at the position of the tryptophan in ATF4, this protein would lose its His-tag, and consequently would end up in the supernatant fraction (S). The +1 and +2 out-of-frame constructs (bottom panel) do not contain a His-tag, whereby the resulting proteins always end up in the supernatant fraction (S) in a His-tag pulldown assay. When frameshifting events take place, however, the His-tag is incorporated into the peptide, whereby the resulting protein ends up in the pulldown fraction (PD). (g) A His-tag pulldown was performed on the lysates of MD55A3 cells expressing the reporters represented in panel 3e. All supernatants (S) and His-tag pulldown samples (PD) of control and IFNγ treated cells were subjected to western blotting and stained with V5 antibodies that invariably detect the N-terminus of all reporter constructs. (h) Western blot analysis showing V5-tagged peptides in pulldown samples of MD55A3 reporter cells, either mock, IFNγ, or IFNγ+IDOi treated. (i) Western blot analysis of V5-tagged proteins in pulldown samples of MD55A3 reporter cells that were either mock-treated or cultured in tryptophan-depleted medium. (j) Western blot analyses showing pulldown assays followed by V5-staining on western blot of MD55A3 cells expressing the original reporter constructs as depicted in FIG. 3e (Wt), or MD55A3 cells expressing the same reporters where the tryptophan codon was mutated to a codon for tyrosine (Y mut). (k) Bar-plots representing the average tGFP signal of IFNγ-treated MD55A3 cells expressing the V5-ATF4(1-63)-tGFP constructs. Values represent the averages +/−SD of three independent flow cytometry experiments, * p<0.05. (1) Anti-VS-tag and anti-tGFP western blot analysis of whole cell lysates from MD55A3 cells expressing the indicated reporters, which were subjected to mock or IFNγ treatments. In each blot the position of the full-length in-frame protein and the shorter out-of-frame protein are marked by the arrowheads. (m) Western blot analyses with anti-V5 antibody of His-tag pulldown samples of 888-Mel and D10 cells expressing the in-frame and +1 reporters. The cells were either grown in isolation (−), or co-cultured with MART-1 specific T cells for 16 hours (+) before the pulldown was performed. (n) Western blot analyses with anti-V5 antibody of His-tag pulldown samples of 888-Mel and D10 that were mock-treated (+), or grown in tryptophan-less medium for 48 hours (−) before the pulldown was performed.

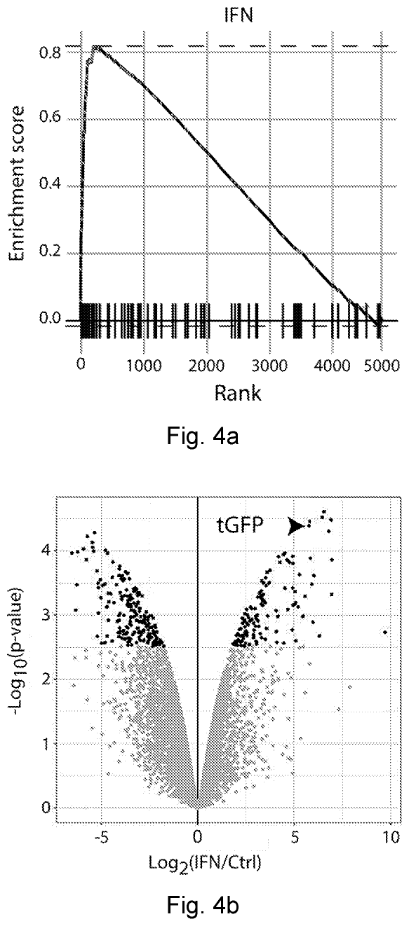

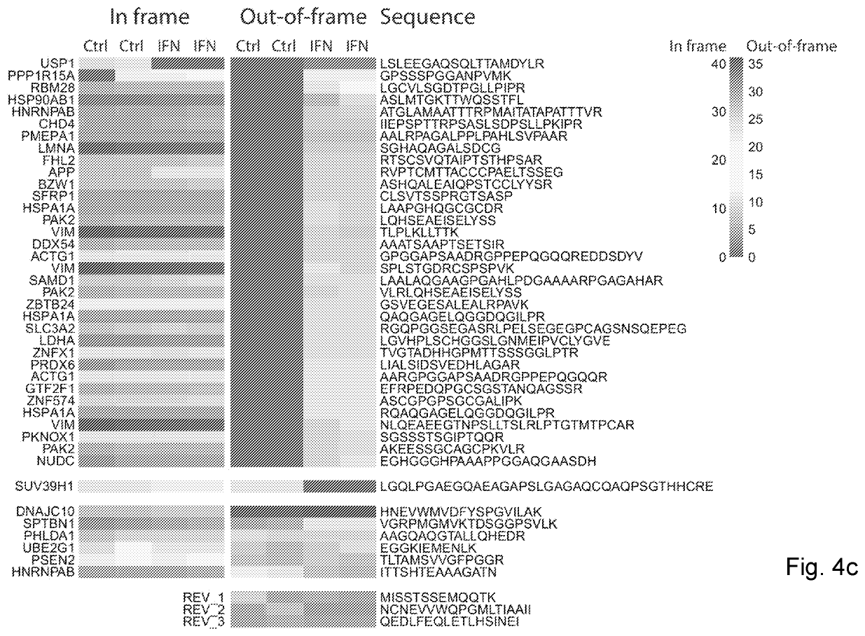

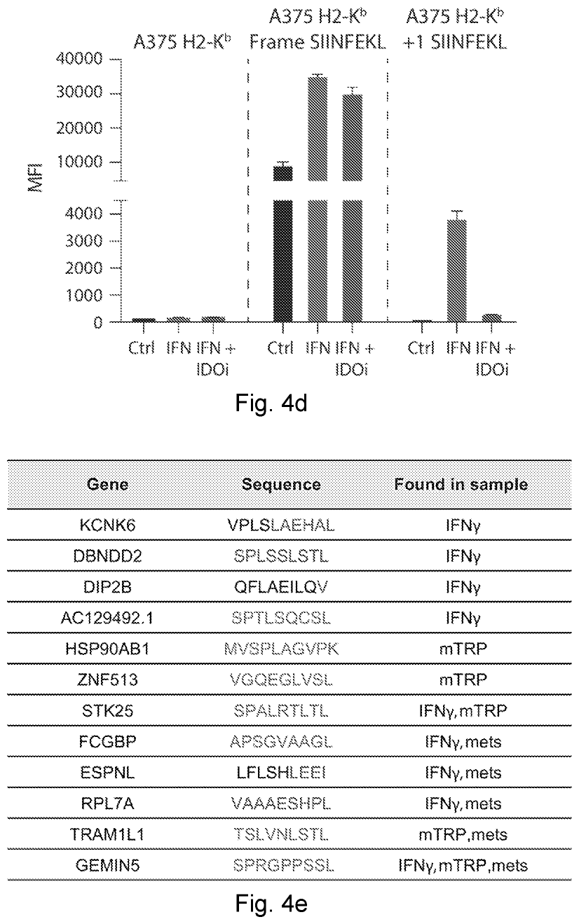

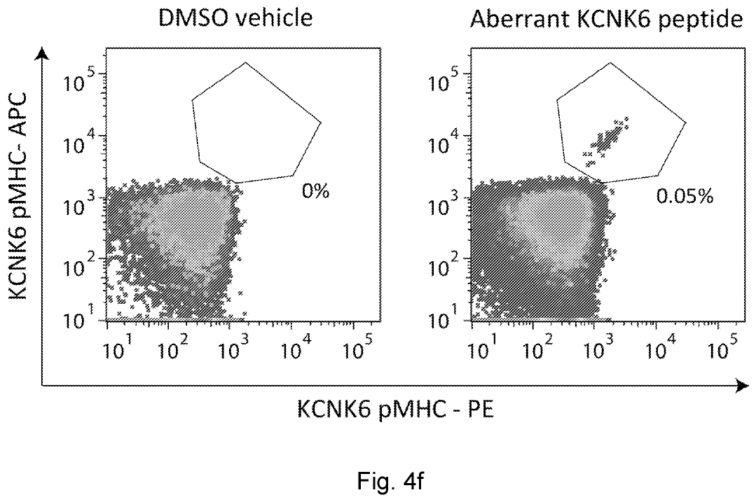

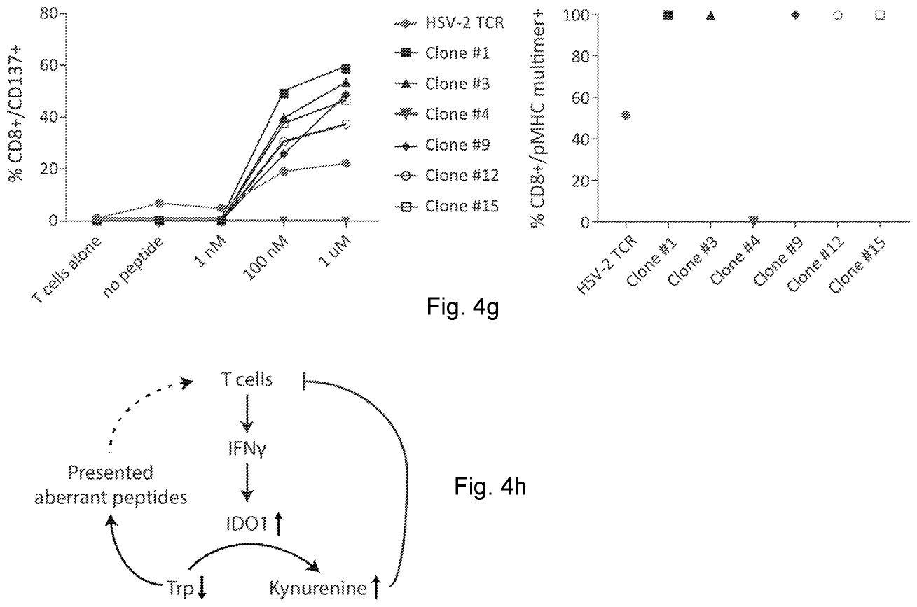

FIG. 4: Aberrant frameshifted peptides are detected in full proteomes, are presented on HLA class I molecules, and induce peptide-specific T cell response.
(a) Gene set enrichment analysis (GSEA) based depiction of the proteomics data showing the induction of the IFN response and HLA genes following IFN treatment of MD55A3 cells. (b) A volcano plot showing differentially identified proteins as detected by 2D LC-MS/MS on lysates of MD55A3 cells expressing the V5-ATF4(1-63)-tGFP +1 out-of-frame construct. The red arrowhead indicates a peptide identified in the full proteome that corresponds to tGFP that could only have arisen from a frameshifting event. (c) Same analysis as panel b, but for the tryptophan-associated aberrant peptides identified to be reproducibly present in either IFNγ-treated or control conditions. REV 1-3 are three peptides identified in a control screen using reverse sequences of the tryptophan-associated aberrant peptides. Note that while 34 out of 41 tryptophan-associated aberrant peptides showed expression only in the IFNγ-treated sample, all three identified control reverse peptides were indifferently expressed in control and IFNγ-treated samples. (d) Bar graph depicting the median fluorescence intensity (MFI) of H2-Kb-bound SIINFEKL peptides in A375 cells expressing H2-Kb in combination with in-frame (Frame) or +1 out-of-frame (+1) V5-ATF4(1-63)-tGFP-SIINFEKL reporters. Bars represent the average of three independent experiments, plus/minus standard deviation. (e) HLA class I presented aberrant peptides. The panel presents 12 frameshifted peptides detected in the immuno-peptidomics mass spectrometry data of IFNγ and tryptophan deprived (mTRP) samples, which were not detected in the untreated controls. A subset of these were also detected in the fresh tumor metastases (mets) samples of the same patient (MD55). The colors of the peptide sequences represent in-frame (black), +1 frame (green) and -1 frame (red). (f) Flow cytometric analysis of CD8+ T cell populations reactive to APC and PE-labeled pMHC multimers complexed with the KCNK6-derived transpeptide in co-cultures of naïve CD8+ T cells and autologous MoDCs pulsed with relevant peptide (right) or DMSO vehicle (left). Cells were from an HLA-B*07:02pos healthy donor. (g) Up-regulation of the activation marker CD137 on T cell clones after co-incubation with K562-B*07:02pos cells pulsed with indicated concentrations of peptides (left). Percentage of T cell clones derived from the sorted KCNK6 pMHC+cells staining positively with APC and PE-labeled KCNK6 pMHC multimers (right). Representative results are shown for five out of 13 reactive, and one out of three non-reactive, T cell clones. T cells transduced with an HSV-2 HLA-B*07:02-restricted TCR were used as a positive control, in the functionality assay when loaded with relevant HSV-2 peptide (left), and for multimer labelling when stained with relevant pMHC multimer (right). (h) A schematic representation of the effects of IFNγ signaling. IFNγ-signaling leads to upregulation of the IDO1 enzyme, which catalyzes the conversion of tryptophan to kynurenine. The increased level of kynurenine inhibits T cell function. But on the other hand, the current study indicates that the IFNγ-induced depletion of tryptophan leads to frameshifting events and the production of aberrant peptides that are presented HLA class I molecules and potentially can reactivate T cells.

### DETAILED DESCRIPTION OF THE INVENTION

### Definitions

The term “T-cell mediated immune response”, as is used herein, refers to protective mechanisms that are responsible for detecting and destroying intracellular pathogens, e.g., cells that are infected with viruses or bacteria. T-cell mediated immune responses can also contribute to the destruction of tumor cells. Key players are CD4+and CD8+ T cells, which produce inflammatory cytokines such as Interferon gamma (IFN-γ) and Tumor Necrosis Factor (TNF). In addition, CD8+ T cells have the ability to induce apoptosis of infected and/or transformed cells.

The term “antigen”, as is used herein, refers to a molecule that can be specifically recognised by the adaptive immune system, that is, by a B cell including antibodies produced by a B cell, or by a T cell. A sequence within an antigen that is bound by an antibody or a T-cell receptor is called an epitope. A preferred antigen comprises one or more epitopes specific for, or highly expressed in, a tumor, including a neo-epitope.

The term “neo-epitope”, as is used herein, refers to an epitope that is not normally present or expressed in a cell such as a tumor cell. Said neo-epitope is a B cell epitope, T cell epitope, or a combination thereof.

A T cell epitope comprises 7-22 amino acid residues, preferably 8-20 amino acid residues, more preferred 8-13 amino acid residues. A preferred antigen is or comprises a polyepitope, comprising 2-50, preferably 5-25 individual epitopes, preferably each contained within a sequence of 8-40 amino acid residues. The individual epitopes in a polyepitope may be alternated by spacer sequences of, preferably, 1-10 amino acid residues.

The term “immune checkpoint inhibitor”, as is used herein, refers to a molecule that blocks an inhibitory interaction between immune cells and other cells or cytokines and which may thereby increase the killing of cancer cells. Examples of checkpoint interacting molecules are PD-1/PD-L1 and CTLA-4/B7-1/B7-2.

A preferred immune checkpoint inhibitor is a molecule that blocks an interaction between PD-1 and PD-Ll. Said molecule that blocks an interaction between PD-1 and PD-L1 preferably is an antibody against PD1 and/or an antibody against PDLL Preferred immune checkpoint inhibitors include a PD1 or PD-L1 blocker such as pembrolizumab (Merck), nivolumab (Bristol-Myers Squibb), pidilizumab (Medivation/Pfizer), MEDI0680 (AMP-514; AstraZeneca) and PDR001 (Novartis); fusion proteins such as a PD-L2 Fc fusion protein (AMP-224; GlaxoSmithKline); atezolizumab (Roche/Genentech), avelumab (Merck/Serono and Pfizer), durvalumab (AstraZeneca), cemiplimab (Regeneron/Sanofi/Genzyme); BMS-936559 (Bristol-Myers Squibb); and small molecule inhibitors such as PD-1/PD-L1 Inhibitor 1 (WO2015034820; (2S)-1-[[2,6-dimethoxy-4-[(2-methyl-3-phenylphenyl)methoxy]phenyl] methyl]piperidine-2-carboxylic acid), BMS202 (PD-1/PD-L1 Inhibitor 2; WO2015034820; N-[2-[[[2-methoxy-6-[(2-methyl[1,1′-biphenyl]-3-yl)methoxy]-3-pyridinyl]methyl]amino] ethyl]-acetamide), and PD-1/PD-L1 Inhibitor 3 (WO/2014/151634; (3S,6S,12S,15S,18S,21S,24S,27S,30R,39S,42S,47aS)-34(1H-imidazol-5-yl)methyl)-12,18-bis((1H-indol-3-yl)methyl)-N,42-bis(2-amino-2-oxoethyl)-36-benzyl-21,24-dibutyl-27-(3-guanidinopropyl)-15-(hydroxymethyl)-6-isobutyl-8,20,23,38,39-pentamethyl-1,4,7,10,13,). Further anti-PD1 molecules include ladiratuzumab vedotin (Seattle Genetics).

An immune checkpoint inhibitor that blocks CTLA4 includes ipilimumab (Bristol-Myers-Squibb).

The term “out of frame peptide”, as is used herein, refers to aberrant peptides that are induced by ribosomal frameshifting at, or downstream of a tryptophan codon, when tryptophan becomes reduced in cells. An out-of-frame peptide normally comprises about 5-40 amino acid residues. A part of said out-of-frame peptide, such as 7-22 amino acid residues, preferably 8-20 amino acid residues, more preferred 8-13 amino acid residues, may become presented by MHC, preferably MHC1, on the surface of the cell.

The term “peptide”, as is used herein, refers to a natural or synthetic compound containing two or more amino acids linked by the carboxyl group of one amino acid to the amino group of another. A peptide preferably encompasses 2-50 amino acid residues.

The term “protein”, as is used herein, refers to a natural or synthetic compound containing two or more amino acids linked by the carboxyl group of one amino acid to the amino group of another. A protein preferably encompasses more than 50 amino acid residues.

The term “reduction of tryptophan”, as is used herein, refers to the reduction of tryptophan in a cell, preferably by deprivation of a cell for tryptophan. Reduction of tryptophan may be accomplished, for example, by providing the cell with growth medium that is depleted of tryptophan, by incubating the cell in the presence of interferon gamma, by activation or expression of indoleamine 2,3-dioxygenase 1 (IDO1), indoleamine 2, 3-dioxygenase 2 (IDO2), and/or tryptophan 2, 3-dioxygenase (TDO) in the cell, or a combination thereof.

The term “tumor cell”, as is used herein, refers to a tumor cell selected from a breast cancer cell; a colon cancer cell; a colorectal cancer cell, especially a microsatellite instability (MSI) high colorectal cancer cell, or a microsatellite instability low, also termed microsatellite stable (MSS) colorectal cancer cell; a bladder cancer cell; a cervical cancer cell; a renal cancer cell; a Hodgkin lymphoma cell; a melanoma cell, including a metastatic melanoma cell; a skin cancer cell; a stomach cancer cell; a hepatocellular cancer cell; a lung cancer cell, including non-small cell lung cancer cell; a head and neck cancer cell; and a kidney tumor cell. The microenvironment of these tumor cells often includes other cell types such as fibroblasts, adipocytes, pericytes, vascular endothelial cells, and, as main players, immune cells. Activation of these immune cells, by inducing an immune response against at least one out-of-frame peptide, may help to reduce or eliminate said tumor cells.

The term “combination”, as is used herein, refers to the administration of effective amounts of an out of frame peptide as defined herein, either as a T cell epitope, a polyepitope, a nucleic acid molecule, and/or a B cell epitope, and interferon gamma and/or reactive T cells, to a patient in need thereof. Said out of frame peptide and interferon gamma and/or reactive T cells may be provided in one pharmaceutical preparation, or as two distinct pharmaceutical preparations. Said combination may be administered to induce an immune response against said out of frame peptide. When administered as two distinct pharmaceutical preparations, they may be administered on the same day or on different days to a patient in need thereof, and using a similar or dissimilar administration protocol, e.g. daily, twice daily, biweekly, orally and/or by infusion. Said combination is preferably administered repeatedly according to a protocol that depends on the patient to be treated (age, weight, treatment history, etc.), which can be determined by a skilled physician. Said induction of an immune response may be prophylactically, meaning that the T cell epitope, polyepitope, nucleic acid molecule, and/or B cell epitope may be administered prior to the administration of interferon gamma and/or reactive T cells, or concurrent with the administration of interferon gamma and/or reactive T cells.

The term “kynureninase (KYNU)”, as is used herein, refers to a pyridoxal-5′-phosphate (pyridoxal-P) dependent enzyme that catalyses cleavage of L-kynurenine and of L-3-hydroxykynurenine into anthranilic acid and 3-hydroxyanthranilic acid, respectively. Alternative splicing results in multiple transcript variants. The human gene encoding KYNU is located on chromosome 2q22.2 and is characterized by HGNC entry code 6469; Entrez Gene entry code 8942, and Ensembl entry code ENSG00000115919. The KYNU protein is characterized by UniProt entry code Q16719.

TABLE 1
Identified out of frame peptides (1-44) and HLA class I
presented aberrant peptides (45-56).

| | | |
|---|---|---|
| 1 | SEQ ID NO: 2 | LSLEEGAQSQLTTAMDYLR |
| 2 | SEQ ID NO: 3 | GPSSSPGGANPVMK |
| 3 | SEQ ID NO: 4 | LGCVLSGDTPGLLPIPR |
| 4 | SEQ ID NO: 5 | ASLMTGKTTWQSSTFL |
| 5 | SEQ ID NO: 6 | ATGLAMAATTTRPMAITATAPATTTVR |
| 6 | SEQ ID NO: 7 | IIEPSPTTRPSASLSDPSLLPKIPR |
| 7 | SEQ ID NO: 8 | AALRPAGALPPLPAHLSVPAAR |
| 8 | SEQ ID NO: 9 | SGHAQAGALSDCG |
| 9 | SEQ ID NO: 10 | RTSCSVQTAIPTSTHPSAR |
| 10 | SEQ ID NO: 11 | RVPTCMTTACCCPAELTSSEG |
| 11 | SEQ ID NO: 12 | ASHCIALEAKWSTCCLYYSR |
| 12 | SEQ ID NO: 13 | CLSVTSSPRGTSASP |
| 13 | SEQ ID NO: 14 | LAAPGHQGCGCDR |
| 14 | SEQ ID NO: 15 | LQHSEAEISELYSS |
| 15 | SEQ ID NO: 16 | TLPLKLLTTK |
| 16 | SEQ ID NO: 17 | AAATSAAPTSETSIR |
| 17 | SEQ ID NO: 18 | GPGGAPSAADRGPPEPQGQQREDDSDYV |
| 18 | SEQ ID NO: 19 | SPLSTGDRCSPSPVK |
| 19 | SEQ ID NO: 20 | LAALAQGAAGPGAHLPDGAAAARPGAGAHAR |
| 20 | SEQ ID NO: 21 | VLRLQHSEAEISELYSS |
| 21 | SEQ ID NO: 22 | GSVEGESALEALRPAVK |
| 22 | SEQ ID NO: 23 | QAQGAGELQGGDQGILPR |
| 23 | SEQ ID NO: 24 | RGQPGGSEGASRLPELSEGEGPCAGSNSQEPEG |
| 24 | SEQ ID NO: 25 | LGVHPLSCHGGSLGNMEIPVCLYGVE |
| 25 | SEQ ID NO: 26 | TVGTADHHGPMTTSSSGGLPTR |
| 26 | SEQ ID NO: 27 | LIALSIDSVEDHLAGAR |
| 27 | SEQ ID NO: 28 | AARGPGGAPSAADRGPPEPQGQQR |
| 28 | SEQ ID NO: 29 | EFRPEDQPGCSGSTANQAGSSR |
| 29 | SEQ ID NO: 30 | ASCGPGPSGCGALIPK |
| 30 | SEQ ID NO: 31 | RQAQGAGELQGGDQGILPR |
| 31 | SEQ ID NO: 32 | NLQEAEEGTNPSLLTSLRLPTGTMTPCAR |
| 32 | SEQ ID NO: 33 | SGSSSTSGPTQQR |
| 33 | SEQ ID NO: 34 | AKEESSGCAGCPKVLR |
| 34 | SEQ ID NO: 35 | EGHGGGHPAAAPPGGAQGAASDH |
| 35 | SEQ ID NO: 36 | LGQLPGAEGQAEAGAPSLGAGAQCQAQPSGTHHCRE |
| 36 | SEQ ID NO: 37 | HNEVWMVDFYSPGVILAK |
| 37 | SEQ ID NO: 38 | VGRPMGMVKTDSGGPSVLK |
| 38 | SEQ ID NO: 39 | AAGQAQGTALLQHEDR |
| 39 | SEQ ID NO: 40 | EGGKIEMENLK |
| 40 | SEQ ID NO: 41 | TLTAMSVVGFPGGR |
| 41 | SEQ ID NO: 42 | ITTSHTEAAAGATN |
| 42 | SEQ ID NO: 43 | MISSTSSEMQQTK |
| 43 | SEQ ID NO: 44 | NCNEVVWQPGMLTAAII |
| 44 | SEQ ID NO: 45 | QEDLFEQLETLHSINEI |
| 45 | SEQ ID NO: 46 | VPLSLAEHAL |
| 46 | SEQ ID NO: 47 | SPLSSLSTL |
| 47 | SEQ ID NO: 48 | QFLAEILQV |
| 48 | SEQ ID NO: 49 | SPTLSQCSL |
| 49 | SEQ ID NO: 50 | MVSPLAGVPK |
| 50 | SEQ ID NO: 51 | VGQEGLVSL |
| 51 | SEQ ID NO: 52 | SPALRTLTL |
| 52 | SEQ ID NO: 53 | APSGVAAGL |
| 53 | SEQ ID NO: 54 | LFLSHLEEI |
| 54 | SEQ ID NO: 55 | VAAAESHPL |
| 55 | SEQ ID NO: 56 | TSLVNLSTL |
| 56 | SEQ ID NO: 57 | SPRGPPSSL |

### Methods of Producing and Identifying an out of Frame Peptide

The invention is based on the surprising finding that a cell, such as a tumor cell, produces at least one out-of-frame peptide of 5-40 amino acid residues as part of a truncated native protein, after reducing or even depleting said cell of tryptophan. Said out-of-frame peptide of 5-40 amino acid residues at the C-terminal end of a truncated protein comprises on average 10-30 amino acid residues, such as about 20 amino acid residues.

Said reduction or depletion may be accomplished by incubating a cell, in vitro or in vivo, in the presence of interferon gamma, and/or by activation of indoleamine 2,3-dioxygenase 1 (IDO1), indoleamine 2, 3-dioxygenase 2 (IDO2), and/or tryptophan 2, 3-dioxygenase (TDO) in the cell. In addition, a low tryptophan diet, or even a tryptophan-free diet, may help in reducing or depleting tryptophan levels in cells. As is known to a person skilled in the art, tryptophan-rich food includes poultry, meat, fish, tofu, beans, lentils, seeds and nuts, oats, caviar, cheese and eggs.

The production of said out of frame peptide is caused by ribosomal bypass of a tryptophan codon in the absence of sufficient levels of tryptophan, which leads to ribosomal frameshifting events. In particular, said peptides may in part be the result of ribosomes that bypassed tryptophan codons by frameshifting events, but then paused with out-of-frame aberrant polypeptides in their lower exit tunnel. Indeed, as is shown in the examples herein below, reporter assays demonstrated the induction of ribosomal frameshifting, and the generation of trans-frame proteins and their presentation at the cell surface after IFNγ treatment. The presence of multiple in frame tryptophan codons within a region of about 8 codons, such as 2 tryptophan codons or three or more tryptophan codons within a region of about 8 codons, results in an increased production of said out of frame peptide.

Said cell, including a tumor cell such as a melanoma cell, that produces an out-of-frame peptide of 5-40 amino acid residues at the C-terminal end of a truncated protein, said peptide comprising on average 10-30 amino acid residues, such as about 20 amino acid residues, may be isolated from an individual that was treated with interferon gamma.

For this, a sample from an individual, preferably comprising tumor cells, may be obtained from a cancerous growth, or of a tumor suspected to be cancerous, depending on the size of the cancerous growth. A cancerous growth can be removed by surgery including, for example, lumpectomy, laparoscopic surgery, colostomy, lobectomy, bilobectomy or pneumonectomy. Said sample can also be derived by biopsy, comprising aspiration biopsy, needle biopsy, incisional biopsy, and excisional biopsy. A sample comprising tumor cells may be obtained from an isolated cancerous growth or part thereof. The act of removing a tumor or part of a tumor is not part of this invention. It is preferred that at least 10% of the cells in the sample are tumor cells, more preferred at least 20%, and most preferred at least 30%. Said percentage of tumor cells can be determined by analysis of a stained section, for example a hematoxylin and eosin—stained section, from the cancerous growth. Said analysis can be performed or confirmed by a pathologist.

As an alternative, said sample comprising tumor cells is obtained from a bodily fluid from an individual. After provision of a bodily fluid from the individual, tumor cells may be enriched, for example, by magnetically separating tumor cells from essentially all other cells in said sample using magnetic nanoparticles comprising antibodies that specifically target said tumor cells.

Part of said out-of-frame peptide of 5-40 amino acid residues may be presented on the surface of the cell by a Major Histocompatibility Complex (MHC). Said presented peptide preferably is 6-15 amino acid residues, more preferably 8-11 amino acid residues, and comprises at least part of the out-of-frame peptide. The presence of said novel epitope, displayed by a MHC molecule, on the surface of a cell, a so called neoepitope, can be used to identify said out of frame peptide.

Said MHC preferably is a MHC-1 molecule, which is expressed by all nucleated cells. T cells that express CD8 molecules react with class I MHC molecules. These T cells often have a cytotoxic function and, therefore may result in lysis of a cell, such as a tumor cell, presenting part of an out-of-frame peptide of 5-40 amino acid residues.

Said MHC-1 molecule was identified as often comprising a Human Leukocyte Antigen (HLA)-DR serotype, such as HLA-DR17 and HLA-DR3, and especially HLA 0301, which occurs frequently in Western Europe, especially in Western Ireland, North of Spain, and Sardinia; a HLA-A24 serotype, and especially HLA 2402, which frequently occurs in Southeastern Asia; or a HLA-A02 serotype, especially HLA 0201, which frequently occurs in the European/North American Caucasian population and is expressed by about half of the individuals.

Said at least one out-of-frame peptide of 5-40 amino acid residues may be identified by proteomics technologies, such as Edman degradation, isotope-coded affinity tag (ICAT) labeling (U.S. Pat. No. 6,670,194), stable isotope labeling with amino acids in cell culture (Ong et al., 2002. Mol Cell Proteomics 1: 376-86), isobaric tag for relative and absolute quantitation (Zieske, 2006. J Exp Bot 57: 1501-1508), and further mass spectrometry (MS)-including techniques, including Liquid Chromatography (LC)—MS, LC-MS-MS, and matrix-assisted laser desorption ionization-time of flight mass spectrometry (MALDI—TOF MS) or even MALDI-TOF/TOF-MS. Developing techniques include nascent fluorescent fingerprinting methods (Timp and Timp, 2020. Science Advances 6: eaax8978) and sub-nanopore arrays for high-throughput single-molecule sequencing of proteins (Lu et al., 2020. View 1: 20200006).

For such comparative analyses, the protein content of cells that were cultured in the presence of normal levels of tryptophan may be compared to the protein content of cells that were cultured in the absence of normal levels of tryptophan, thus after reducing or even depleting said cells of tryptophan. Proteins may firstly be digested, followed by fractionation of the digested peptide mixture and MS-analysis of the fractionated peptides, for example in an LC-MS/MS configuration. Said proteins may include all cytoplasmic proteins, or a subset of protein that are expressed on the cell surface.

### Products of the Invention

The invention provides a peptide of 8-25 amino acid residues, preferably 8-22 amino acid residues, more preferred 8-13 amino acid residues according to the invention that is obtainable upon reduction or depletion of tryptophan in a cell, for example by treating the cell with interferon gamma and/or by activation of indoleamine 2,3-dioxygenase 1 (IDO1), indoleamine 2, 3-dioxygenase 2 (IDO2), and/or tryptophan 2, 3-dioxygenase (TDO) in the cell, and analyzing the display of neoepitopes by MHC on the surface of the cells. Said peptides are derived from larger peptides that are generated by ribosomal bypass of a tryptophan codon in the absence of sufficient levels of tryptophan, which result in ribosomal frameshifting events. The presence of multiple in frame tryptophan codons within a region of about 8 codons, such as 2 tryptophan codons or three or more tryptophan codons within a region of about 8 codons, results in an increased production of said larger peptides. These larger peptides are not encoded by the normally used reading frame. Hence, part of these larger peptides may be exposed as by MHC on the surface of the cells as a non-self peptide. Said peptide of 8-25 amino acid residues, therefore, is in fact a T cell epitope that is or can be exposed by MHC on the surface of the cells as a non-self peptide.

The invention further provides an out-of-frame peptide of 5-40 amino acid residues that is produced by a cell upon reduction of tryptophan in said cell.

A preferred out-of-frame peptide of 5-40 amino acid residues that is produced by a cell upon reduction or depletion of tryptophan in said cell, is selected from Table 1. As is indicated in Table 1, peptides # 45-56 were presented by MHC-1 on the surface following reduction or depletion of tryptophan in said cells, and can be used for immunotherapy.

Further preferred peptides that are presented by a triple negative breast cancer cell line such as MDA-MB-231 include TLVEDLLEV (SEQ ID NO:58; RFX1), LLTHGLLLL (SEQ ID NO:59; ILRUN), LLEGLLTTI (SEQ ID NO:60; ETF1), TVIGTLLEI (SEQ ID NO:61; RHOBTB3), GLLETHPALLL (SEQ ID NO:62; SRSF3), and LMSLHLVHLPSQLTC (SEQ ID NO:63; CNIH4). These peptides are likely displayed by HLA-A*02:01, which is the most abundant allele in Europe and among not Asian populations. SEQ ID NO:58, SEQ ID NO:60, SEQ ID NO:61, and SEQ ID NO:63 showed high mRNA expression, but little protein expression in these cells. Hence, these non-self peptides are likely derived from highly degraded proteins. Said peptides are presented as non-self peptides by MHC on the surface of the cells.

SEQ ID NO:61, SEQ ID NO:58 and SEQ ID NO:62 were tested and showed immunogenicity in peripheral blood mononuclear cells from healthy donors.

A most preferred peptide that is generated by cells upon reduction of tryptophan and that is exposed by MHC molecules at the surface of said cells is TVIGTLLEI (SEQ ID NO:61; RHOBTB3). Further preferred peptides include TLVEDLLEV (SEQ ID NO:58; RFX1) and GLLETHPALLL (SEQ ID NO:62; SRSF3). Even further preferred peptides are LLTHGLLLL (SEQ ID NO:59; ILRUN), LLEGLLTTI (SEQ ID NO:60; ETF1), TVIGTLLEI (SEQ ID NO:61; RHOBTB3), and LMSLHLVHLPSQLTC (SEQ ID NO:63; CNIH4), and SEQ ID NOs 45-56.

The invention further provides a T cell epitope comprising 8-22 amino acid residues, more preferred 8-13 amino acid residues, of an out-of-frame peptide according to the invention. A preferred T cell epitope is selected from peptides # 45-56 of Table 1, and SEQ ID NOs 58-63. Said T cell epitopes can be used to stimulate an immune response in an individual, such as an individual that is suffering from a tumor and who will be treated with interferon gamma.

A T cell epitope according to the invention preferably is provided as a polyepitope, comprising 2-50, preferably 5-25 individual T cell epitopes according to the invention. Said individual T cell epitopes preferably are each contained within a sequence of 8-40 amino acid residues. Said individual T cell epitopes may be alternated by spacer sequences, preferably of 1-10 amino acid residues.

The invention further provides a B cell epitope comprising at least one an out-of-frame peptide according to the invention. A preferred B cell epitope is selected from peptides # 01-56 of Table 1, SEQ ID NOs 58-63, and combinations thereof. Said B cell epitopes can be used to stimulate an immune response in an individual, such as an individual that is suffering from a tumor and who is or will be treated with interferon gamma.

The invention further provides a nucleic acid molecule encoding a B cell epitope, a T cell epitope according to the invention, or an polyepitope according to the invention. Said nucleic acid molecule preferably is a RNA molecule, or a DNA molecule that expresses said polyepitope upon delivery to a suitable cell.

A nucleic acid molecule according to the invention preferably is provided as an expression construct that expresses said nucleic acid molecule in a cell of interest. Said expression construct may be chosen from a plasmid and a viral vector such as a retroviral vector. Said viral vector preferably is a recombinant adeno-associated viral vector, a herpes simplex virus-based vector, or a lentivirus-based vector such as a human immunodeficiency virus-based vector. Said viral vector most preferably is a retroviral-based vector such as a lentivirus-based vector such as a human immunodeficiency virus-based vector, or a gamma-retrovirus-based vector such as a vector based on Moloney Murine Leukemia Virus (MoMLV), Spleen-Focus Forming Virus (SFFV), Myeloproliferative Sarcoma Virus (MPSV) or on Murine Stem Cell Virus (MSCV). A preferred retroviral vector is the SFG gamma retroviral vector (Riviere et al., 1995. PNAS 92: 6733-6737).

Retroviruses, including a gamma-retrovirus-based vector, may be packaged in a suitable complementing cell that provides Group Antigens polyprotein (Gag)-Polymerase (Pol) and/or Envelop (Env) proteins. Suitable packaging cells are human embryonic kidney derived 293T cells, Phoenix cells (Swift et al., 2001. Curr Protoc Immunol, Chapter 10: Unit 10 17C) or Flp293A cells (Schucht et al., 2006. Mol Ther 14: 285-92).

Said vector may be a plasmid such as pCMV and pcDNA or, preferably, a viral vector. Said vector preferably comprises a promoter for expression of the protein of interest in a suitable host cell. Said promoter may be a constitutive promoter or an inducible promoter, and may provide low, medium or high expression levels of the nucleic acid molecule.

As an alternative, said nucleic acid molecule may be provided as a non-replicating nucleic acid molecule, which may be packaged and delivered to an individual in need thereof, as an in vivo self-replicating nucleic acid molecule, which may be packaged with additional nucleic acid strands that ensure it will be copied once the nucleic acid molecule is inside a cell, or as an in vitro dendritic cell, which may be extracted from the individual's blood, transfected with the nucleic acid molecule, then returned to the patient to stimulate an immune reaction.

Further alternatives are provided by nude nucleic acid molecules, or liposomes, polymerizers and molecular conjugates that comprise the nucleic acid molecule. Minicircle DNA vectors free of plasmid bacterial DNA sequences may be generated in bacteria and may express said nucleic acid molecule at high levels in vivo.

Said cell of interest may be an antigen presenting cell that expresses MHC type II, such as a dendritic cell, a mononuclear phagocyte, and a B cell. These cells are important in initiating immune responses. Said cell of interest may be an autologous cell that has been isolated from an individual, provided with a nucleic acid molecule encoding a T cell epitope according to the invention, or an polyepitope according to the invention, and returned back to the individual. As an alternative, said cell of interest is a generic cell, such as for example a DCOne® cell (DCPrime; Leiden, the Netherlands), which is derived from myeloid leukemia cells and expresses a number of validated tumor antigens.

The invention further provides a T cell, comprising a T cell Receptor (TCR) that is directed against a T cell epitope according to the invention. Said TCR preferably is an αβTCR. Methods to isolate T cells that bind to a T cell epitope according to the invention are known in the art. Said T cells can be isolated from an individual that has been treated with interferon gamma, especially an individual that has suffered from a tumor such as a melanoma or a breast cancer. As an alternative, said T cells may be generated by expressing a reactive TCR that is directed against a T cell epitope according to the invention in T cells, for example by recombinant means. Said T cells may by autologous T cells, i.e. derived from the patient suffering from a tumor, or heterologous T cells.

### Methods of Treatment

The invention further provides a method of inducing an immune response in an individual against at least one out-of-frame peptide of 5-40 amino acid residues that is produced by a cell, said method comprising providing said individual with a T cell epitope according to the invention, a polyepitope according to the invention, a nucleic acid molecule according to the invention, or a combination thereof. Said method of inducing an immune response especially can be performed prophylactically prior to, or in combination with, treatment of the individual with interferon gamma. Treatment of the individual, for example with interferon gamma, may induce the generation of at least one out-of-frame peptide of 5-40 amino acid residues in a cell, which cell will be targeted by the induced immune response. As an alternative, or in addition, a low tryptophan diet, or even a tryptophan-free diet, may help in generating at least one out-of-frame peptide of 5-40 amino acid residues in a cell that can be targeted by the induced immune response.

The invention further provides a method of treating an individual suffering from a tumor such as a melanoma or a breast cancer, comprising providing said individual with a T cell epitope according to the invention, a polyepitope according to the invention, a nucleic acid molecule according to the invention, a T cell according to the invention, or a combination thereof. Said individual has been, or is being treated with interferon gamma and is likely to comprise a tumor cell such as a melanoma cell that expresses the at least one out-of-frame peptide of 5-40 amino acid residues. The induction of an immune response in the individual by providing said individual with a T cell epitope according to the invention, a polyepitope according to the invention, a nucleic acid molecule according to the invention, and/or the provision to the individual of a T cell according to the invention that aids in killing cells, especially tumor cells, that express an out-of-frame peptide of 5-40 amino acid residues in a cell and, preferably, express a T cell epitope comprising 8-22 amino acid residues, more preferred 8-13 amino acid residues, of an out-of-frame peptide according to the invention, will aid in the treatment of the individual.

In one embodiment, said provision of an individual with a T cell epitope, a polyepitope, a B cell epitope, a nucleic acid molecule, or a combination thereof, according to the invention can be performed by providing the individual with a peptide or protein encompassing said T cell epitope, polyepitope, or B cell epitope.

Said peptide or protein encompassing said T cell epitope, polyepitope, B cell epitope, or combination thereof, may be produced by chemical synthesis, including an automated chemistry platform such as described in Hartrampf et al., 2020. (Hartrampf et al., 2020. Science 368: 980-987).

Said peptide or protein encompassing said T cell epitope, polyepitope, B cell epitope, or combination thereof, may further be expressed and purified from a suitable expression system. Commonly used expression systems for heterologous protein production include E. coli, Bacillus spp., baculovirus, yeast, fungi such as filamentous fungi and yeasts such as Saccharomyces cerevisiae and Pichia pastoris, eukaryotic cells such as Chinese Hamster Ovary cells (CHO), human embryonic kidney (HEK) cells and PER.C6® cells (Thermo Fisher Scientific, MA, USA), and plants.

For production of a peptide or protein encompassing said T cell epitope, polyepitope, B cell epitope, or combination thereof, an expression construct, preferably DNA, may be produced by recombinant technologies, including the use of polymerases, restriction enzymes, and ligases, as is known to a skilled person. Alternatively, said expression construct is provided by artificial gene synthesis, for example by synthesis of partially or completely overlapping oligonucleotides, or by a combination of organic chemistry and recombinant technologies, as is known to the skilled person. Said expression construct preferably is a vector that is able to direct expression of an open reading frame that is operatively-linked to suitable regulatory elements. Said suitable regulatory elements include promoters, enhancers, internal ribosomal entry sites (IRES), and other expression control elements such as a 5′ untranslated region, a 3′ untranslated region and, optionally, transcription termination signals such as a polyadenylation signal. Regulatory elements include elements that provide direct constitutive expression in many cell types and elements that direct expression of the nucleotide sequence only in certain cells (i.e., tissue-specific regulatory sequences). Regulatory elements may also direct expression in a temporal-dependent manner, such as in a cell-cycle dependent or developmental stage-dependent manner, which may or may not also be tissue or cell-type specific. Examples of suitable promoters include pol II promoters such as retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the b-acting promoter, the phospho-glycerol kinase (PGK) promoter, and the EF1a promoter. As well as promoters, regulatory elements may include enhancer elements, such as WPRE; CMV enhancers; the R-U5′ segment in LTR of HTLV-I; SV40 enhancer; and the intron sequence between exons 2 and 3 of rabbit b-globin. It will be appreciated by those skilled in the art that the design of the expression vector can depend on such factors as the choice of the host cell to be transformed, the level of desired expression etc. Said regulatory elements such as promoter sequences may be an autologous sequences, or heterologous sequences, i.e. derived from a different species.

The efficiency of expression of recombinant proteins in a heterologous system depends on many factors, both on the transcriptional level and the translational level. For example, said expression construct may be codon-optimized to enhance expression in a cell of interest, such as E. coli. Further optimization may include the removal of cryptic splice sites, removal of cryptic polyA tails and/or removal of sequences that may lead to unfavorable folding of the mRNA. In addition, the expression construct may encode a protein export signal for secretion of the peptide or protein out of the cell, allowing efficient purification of the peptide or protein.

Methods for purification of peptides and/or proteins are known in the art and are generally based on chromatography such as affinity chromatography and ion exchange chromatography, to remove contaminants. In addition to contaminants, it may also be necessary to remove undesirable derivatives of the product itself such as degradation products and aggregates. Suitable purification process steps are, for example, provided in Berthold and Walter, 1994 (Berthold and Walter, 1994. Biologicals 22: 135-150).

As an alternative, or in addition, a recombinant peptide or protein may be tagged with one or more specific tags by genetic engineering to allow attachment of the protein to a column that is specific to the tag and therefore be isolated from impurities. The purified protein is then exchanged from the affinity column with a decoupling reagent. The method has been routinely applied for purifying recombinant protein. Conventional tags for proteins, such as histidine tag, are used with an affinity column that specifically captures the tag (e.g., a Ni-IDA column for the histidine tag) to isolate the protein from other impurities. The peptide or protein may then be exchanged from the column using a decoupling reagent according to the specific tag (e.g., imidazole for histidine tag). Suitable tags include one or more of a c-myc domain (EQKLISEEDL; SEQ ID NO:64), a hemagglutinin tag (YPYDVPDYA; SEQ ID NO:65), a maltose-binding protein, glutathione-S-transferase, a FLAG tag peptide, biotin acceptor peptide, streptavidin-binding peptide and calmodulin-binding peptide, as presented in Chatterjee, 2006 (Chatterjee, 2006. Cur Opin Biotech 17, 353-358). Methods for employing these tags are known in the art and may be used for purifying a Cas protein or proteins. When present, said tag can preferably be cleaved from the peptide or protein before providing an individual with the peptide or protein encompassing said T cell epitope or polyepitope.

In an embodiment, said provision of an individual with a T cell epitope, a polyepitope, a B cell epitope, a nucleic acid molecule, or a combination thereof, according to the invention can be performed by providing the individual with a nucleic acid molecule according to the invention encoding said T cell epitope, polyepitope, or B cell epitope.

In an embodiment, said nucleic acid molecule is provided in a vector, especially in a viral vector such as an adeno-associated viral vector, a lentiviral vector, or a herpes simplex virus vector, to deliver the nucleic acid molecule in a relevant cell of an individual. Said viral vector preferably provides temporal expression of the nucleic acid molecule. Said viral vector preferably is a recombinant adenovirus-based vector, an alphavirus-based vector, a herpes simplex virus-based vector, or a pox virus-based vector. Said viral vector most preferably is a adenoviral-based vector or a self-amplifying alphavirus-based replicon vector (Ljungberg and Liljeström, 2015. Expert Rev Vaccines 14: 177-194).

Said nucleic acid molecule may also be provided as a DNA molecule that expresses said polyepitope upon delivery to a suitable cell. Said DNA molecule may comprise modified nucleotides, for example to increase half-life of the molecule. For example, said nucleic acid molecule may be provided in a plasmid, or as linear DNA. Non-virus mediated delivery of a DNA molecule according to the invention include lipofection, microinjection, and agent-enhanced uptake of DNA. Lipofection is described in e.g., U.S. Pat. Nos. 5,049,386, 4,946,787; and 4,897,355) and lipofection reagents are sold commercially (e.g., Transfectam™, Lipofectin™, and SAINT™). Cationic and neutral lipids that are suitable for efficient lipofection of polynucleotides include those of WO 91/17424 and WO 91/16024. Delivery can be to cells (e.g. in vitro or ex vivo administration) or to target tissues (e.g. in vivo administration. Said DNA molecule may also be packaged, for example in a virosome, a liposome, or immunoliposome, prior to delivery of said DNA molecule to an individual in need thereof.

Said nucleic acid molecule may also be provided as a RNA molecule that expresses said T cell epitope, polyepitope and/or B cell epitope, upon delivery to a suitable cell. Said RNA molecule may be synthesized in vitro, for example by a DNA dependent RNA polymerase such as T7 polymerase, T3 polymerase, SP6 polymerase, or a variant thereof. Such variant may include for instance a mutant T7 RNA polymerase that is capable of utilizing both canonical and non-canonical ribonucleotides and deoxynucleotides as substrates (Kostyuk et al., 1995. FEBS Lett. 369: 165-168; Sousa et al., 1995. EMBO J. 14(18): 4609-4621; Gudima et al., 1998. FEBS Lett. 439: 302-306; Padilla et al., 2002. Nucl. Acids Res. 30(24): e138), a RNA polymerase variant displaying higher thermostability such as Hi-T7™ RNA Polymerase from New England Biolabs (Boulain et al., 2013. Protein Eng Des Sel. 26(11): 725-734), or a mutant RNA polymerase with decreased promoter specificity (Ikeda et al., 1993. Biochemistry 32(35):9115-9124).

Said RNA molecule may encompass, for example, a synthetic cap analogue (Stepinski et al., 2001. RNA 7: 1486-1495), one or more regulatory elements in the region (UTR) and/or the 3′-UTR that stabilize said RNA molecule and/or increases protein translation (Ross and Sullivan, 1985. Blood 66: 1149-1154), and/or modified nucleosides to increase stability and/or translation (Kariko et al., 2008. Mol Ther 16: 1833-1840), and/or to decrease an inflammatory response (Kariko et al., 2005. Immunity 23: 165-175). (2005). In addition, said RNA molecule preferably encompasses a poly(A) tail to stabilize the RNA molecule and/or to increase protein translation (Gallie, 1991. Genes Dev 5: 2108-2116). In addition, said RNA molecule preferably is codon optimized to increase translation. Codon optimization is offered by commercial institutions, such as ThermoFisher Scientific, called Invitrogen GeneArt Gene Synthesis, GenScript, called GenSmart™ Codon Optimization, or GENEWIZ, called GENEWIZ's codon optimization tool.

Further factors that may increase the induction of an immune response against the T cell epitope, polyepitope and/or B cell epitope after provision of an RNA molecule to an individual in need thereof include co-delivery of translation initiation factors such as, for example, the eukaryotic translation initiation factor 4E.

Said RNA molecule may be delivered to an individual ex vivo, for example by loading said RNA molecule into dendritic cells followed introducing the cells to an individual in need thereof, for example by infusion, or by parenteral administration.

Said RNA molecule may be delivered to an individual in the presence or absence of a carrier. Said carrier preferably allows prolonged expression in vivo of the T cell antigen or polyepitope. Said carrier may be one or more of a cationic protein such as protamine, a protamine liposome, a polysaccharide, a cation, a cationic polymer, a cationic lipid, cholesterol, polyethylene glycol, and a dendrimer. For example, said RNA molecule may be delivered as a naked RNA molecule, complexed with protamine, associated with a positively charged oil-in-water cationic nanoemulsion, associated with a chemically modified dendrimer and complexed with polyethylene glycol (PEG)-lipid, complexed with protamine in a PEG-lipid nanop article, associated with a cationic polymer such as polyethylenimine, associated with a cationic polymer such as PEI and a lipid component, or associated with a polysaccharide such as, for example, chitosan, in a cationic lipid nanoparticle such as, for example, 1,2-dioleoyloxy-3-trimethylammoniumpropane (DOTAP) or dioleoylphosphatidylethanolamine (DOPE) lipids), complexed with cationic lipids and cholesterol, and complexed with cationic lipids, cholesterol and PEG-lipid, as is described in Pardi et al., 2018. Nature Reviews 17: 261-279).

A carrier may further comprise one or more RNAs that encode immune activator proteins such as a member of the Tumor Necrosis Factor (Ligand) Superfamily, for example CD70 and/or CD40 ligand, and constitutively active Toll-like Receptor 4 (Van Lint et al., 2012. Cancer Res 72: 1661-1671).

Said nucleic acid molecule encoding a T cell epitope, a polyepitope and/or a B cell epitope, may be administered by a parenteral route, including subcutaneous, intradermal, intramuscular, intravenous, intralymphatic, intranodal administration. As is known to a person skilled in the art, a carrier may be selected to is suited for a specific mode of administration in order to achieve a desirable outcome. For example, a mucoadhesive carrier with hydrophilic surfaces have been used to target nasal-associated lymphoid tissue to overcome impediments such as poor tissue permeability and mucociliary clearance in the nose (Jahanafrooz et al., 2020. Drug Discovery Today 25: 552-560).

Said individual may further be provided with interferon gamma, an immune checkpoint inhibitor, or both, whereby said interferon gamma and/or immune checkpoint inhibitor may be administered prior to, simultaneously with, or following administration of a T cell epitope according to the invention, a polyepitope according to the invention, a nucleic acid molecule according to the invention, a T cell according to the invention, or a combination thereof.

A preferred combination includes the provision of reactive T cells that are directed against the aberrant out-of-frame peptides, in combination with a nucleic acid molecule such as a RNA molecule that expresses said T cell epitope, polyepitope and/or B cell epitope, upon delivery to a suitable cell, in order to boost said antitumor immunity.

Said immune checkpoint inhibitor preferably is administered intravenously, preferably by infusion. Said immune checkpoint inhibitor preferably is administered once every 2-4 weeks for a period of 1-24 weeks. The preferred dosage of selected immune checkpoint inhibitors is 2-4 mg/kg. preferably about 3 mg/kg every 2-4 weeks, or 240-480 mg every 2-4 weeks for ipilimumab; 100-400 mg, preferably about 200 mg every 2-4 weeks, preferably every 3 weeks for pembrolizumab; 100-500 mg, preferably 240-480 mg every 2-4 weeks, preferably every 2 weeks for nivolumab; 2-12 mg/kg. preferably 4-8 mg/kg every 2-4 weeks, preferably every 4 weeks for pidilizumab; 100-500 mg, preferably about 350 mg every 2-4 weeks, preferably every 3 weeks for cemiplimab; 600-1800 mg, preferably about 1200 mg every 2-4 weeks, preferably every 3 weeks for atezolizumab; 400-1200 mg, preferably about 800 mg, every 2-4 weeks, preferably every 2 weeks for avelumab; and 5-15 mg/kg, preferably about 10 mg/kg, or 1000-2000 mg, preferably about 1500 mg, every 2-4 weeks, preferably every 2 weeks for durvalumab. A person skilled in the art will understand that the dosage in a combination with a according to the invention, may be at the low range of the indicated dosages, or even below the indicated dosages.

Said individual may additionally be provided with an inducer of kynureninase, such as a kynureninase expression construct. Said kynureninase expression construct preferably comprises a human kynureninase, preferably a human kynureninase with RefSeq accession number NM_003937.3 or a splice variant or functional part thereof. Said expression construct may be a nuclei acid molecule, a plasmid, or a viral vector, as is described herein above. A suitable expression construct, pcDNA-KYNU, is commercially available from OriGene (#RC214932).

The provision of an inducer of kynureninase may aid in suppressing tumor cell proliferation and may aid in activating immune cells of the individual to react with and to kill the tumor cells.

The invention further provides a pharmaceutical composition, comprising a T cell epitope according to the invention, a polyepitope according to the invention, a B cell epitope according to the invention, a nucleic acid molecule according to the invention, a T cell according to the invention, or a combination thereof and, optionally, an accessory molecule such as an adjuvant, an immune checkpoint inhibitor, an immune stimulating molecule such as a chemokine and/or a cytokine, an inducer of kynureninase, or a combination thereof.

The invention further provides a method of treating a tumor in a subject, the method comprising the simultaneous, separate or sequential administering to the subject of effective amounts of an out of frame peptide as defined herein, either as a T cell epitope, a polyepitope, a B cell epitope, and/or a nucleic acid molecule, and interferon gamma, to a subject in need thereof. Said combination of an out of frame peptide as defined herein, either as a T cell epitope, a polyepitope, a B cell epitope, and/or a nucleic acid molecule, and interferon gamma preferably further comprises an immune checkpoint inhibitor.

Said combination of an out of frame peptide as defined herein, either as a T cell epitope, a B cell epitope, a polyepitope, and/or a nucleic acid molecule, and interferon gamma, optionally also including an immune checkpoint inhibitor, either separately or in combination, may be administered by oral administration, topical administration, nasal administration, inhalation, topical, transdermal and/or parenteral administration, including intramuscular, subcutaneous, intraperitoneal administration. A preferred mode of administration is oral administration and/or parenteral administration such as intravenous and/or subcutaneous administration. For oral administration, a preferred pharmaceutical preparation is provided by a tablet.

Said interferon gamma preferably is subcutaneously administered at 10-100 microgram/m², such as 20-80 microgram/m², including about 50 microgram/m², or at 0.5-5 microgram per kilogram body weight, such as about 1.5 microgram/kg. The administration of interferon gamma is preferably performed at regular intervals, such as weekly, twice weekly or 3 times weekly. Interferon gamma can be injected by the patient or caregiver after appropriate training.

Pharmaceutically acceptable excipients include diluents, binders or granulating ingredients, a carbohydrate such as starch, a starch derivative such as starch acetate and/or maltodextrin, a polyol such as xylitol, sorbitol and/or mannitol, lactose such as α-lactose monohydrate, anhydrous α-lactose, anhydrous β-lactose, spray-dried lactose, and/or agglomerated lactose, a sugar such as dextrose, maltose, dextrate and/or inulin, or combinations thereof, glidants (flow aids) and lubricants to ensure efficient tableting, and sweeteners or flavours to enhance taste.

### EXAMPLES

### Example 1

### Materials and Methods

Alignment of Ribosome Profiling Data

Preprocessing of FASTQ files consisted of adapter removal using cutadapt (available at doi.org/10.14806/ej.17.1.200) with the parameters (--quality-base=33-O7-e 0.15-m 20-q 5) and removal of rRNA and tRNA contaminants by means of alignment against a reference (rRNA reference data from GENCODE⁶¹ v19: rRNA, MT_RNA, rRNA_pseudogene, and tRNA reference data from GtRNAdb) using bowtie2⁶² with parameters (--seed 42−p1—local).

The actual alignment of pre-processed FASTQ files was done with TopHat2⁶³ and Bowtie2⁶² against GRCh37/ hg19 and GENCODE v19/BASIC transcript with Ensembl coordinates using parameters (seed 42-n 2-m 1 --no-novel-juncs --no-novel-indels --no-coverage-search --segment-length 25). In a subsequent step the primary aligned reads filtered for a minimum mapping quality of 10.

Quality check of the FASTQ files were undertaken using FASTQC package (ref), and the quality analysis of frames and periodicity of RPFs was undertaken using RiboWaltz⁶⁴ (data not shown).

Diricore Analysis

For the subsequence analysis RPF codon occupancy frequency between two conditions (e.g. plus IFNγ versus minus IFNγ) are compared as described in 21. RPF density analysis is performed by the comparison of normalized 5′ RPF density analysis per codon between conditions²¹.

RNA-Seq Data Analysis

RNA-seq data, as FASTQ file, were aligned to human hg19 genome using TopHat⁶³. SAMTOOLS⁶⁵ was used for file format conversions. HTSeq⁶⁶ was used to count reads at exons of protein coding genes. Library size normalization of read counts was done using DESEQ⁶⁷.

Finding Bumps in the Ribosome Profiling Data and Associating them with Amino-Acids

The reads (FASTQ) from Riboseq experiments were aligned to human transcript assembly (gencode v19) after removal of low quality reads and the reads that align to tRNA and rRNA (refer methods section—Diricore). Transcript alignment was performed using Bowtie 62 with the default parameters.

BAM files were converted to BED using BEDTOOLS 68 and later file formats were edited using PERL scripts. Each gene was divided into 100 windows of equal length, and read (separately for every sample) at each window were quantified using BEDTOOLS. The array of reads, logged (base 2), were normalized between 0 to 1. For this, the replicates for both the conditions (untreated and IFNγ treated conditions) were taken collectively as average. Thereafter, PEAKS were identified in an array of reads per window using findpeaks (pracma v1.9.9) function in R with (nups=1, ndown=1, minpeakheight=10) parameters.

Only peaks called in treatment condition (merged in two replicates) were identified as TREATMENT (IFNγ-treated) peaks, while peaks called in minus condition were identified as CONTROL peaks. The highest point (window with the greatest number of reads) per peak, was marked as reference point.

The transcript position of the reference point, was converted to protein coordinate using ensembldb 2.8.0⁶⁹ in R. The amino-acids were mapped 30 codons at each side of the reference point and was quantified as sum at every individual position in PERL. Line plots were then plotted in R. Upstream/Downstream ratio for every amino-acid was quantified as the ratio of average presence of a particular amino acid upstream (30 codons) upstream versus 30 codons downstream. The scripts are available with additional detailing in the github package (see github.com/apataskar/bump_finder_example2).

Transcript density plots, are plotted as the function of density (R function) of read across the nearest “tryptophan” to the reference-points identified in the respective cell-lines. For global Bump-analysis, transcript positions of TGG codon encoding tryptophan amino-acid in-frame were obtained using customized PERL script. The frequency of occurrence of P-sites (12th position from offset of the read) from Ribosome profiling samples across 30 codons (upstream and downstream) were plotted as density function in R and as heatmap using pheatmap in R.

Proteomics Analysis (Relates to FIG. 2l-s)

I) Sample Preparation for Proteomics

Frozen MD55A3 cell pellets were lysed, reduced and alkylated in heated guanidine (GuHCl) lysis buffer as described by Jersie-Christensen et al.⁷⁰. Proteins were digested with Lys-C (Wako) for 2 h at 37° C., enzyme/substrate ratio 1:100. The mixture was then diluted to 2 M GuHCl and digested overnight at 37° C. with trypsin (Sigma) in enzyme/substrate ratio 1:50. Digestion was quenched by the addition of TFA (final concentration 1%), after which the peptides were desalted on a Sep-Pak C18 cartridge (Waters, Massachusetts, USA). Samples were vacuum dried and stored at −80° C. until LC-MS/MS analysis.

Peptides were reconstituted in 2% formic acid and analyzed by nano LC-MS/MS on an Q Exactive HF-X Hybrid Quadrupole-Orbitrap Mass Spectrometer equipped with an EASY-NLC 1200 system (Thermo Scientific). Samples were directly loaded onto the analytical column (ReproSil-Pur 120 C18-AQ, 2.4 μm, 75 μm×500 mm, packed in-house). Solvent A was 0.1% formic acid/water and solvent B was 0.1% formic acid/80% acetonitrile. Peptides were eluted from the analytical column at a constant flow of 250 nl/min. For single-run proteome analysis, a 3 h gradient was employed containing a linear increase from 4% to 26% solvent B, followed by a 15-min wash. MS settings were as follows: full MS scans (375-1500 m/z) were acquired at 60,000 resolution with an AGC target of 3×106 charges and max injection time of 45 ms. Loop count was set to 20 and only precursors with charge state 2-7 were sampled for MS2 using 15,000 resolution, MS2 isolation window of 1.4 m/z, 1×10⁵ AGC target, a max injection time of 22 ms and a normalized collision energy of 26.

II) Data Analysis

RAW files were analyzed by Proteome Discoverer (version 2.3.0.523, Thermo Scientific) using standard settings. MS/MS data were searched in Sequest HT against the the human Swissprot database (20,381 entries, release 2018_08). The maximum allowed precursor mass tolerance was 50 ppm and 0.06 Da for fragment ion masses. False discovery rates for peptide and protein identification were set to 1%. Trypsin was chosen as cleavage specificity allowing two missed cleavages. Carbamidomethylation (C) was set as fixed modification, whereas oxidation (M) and protein N-terminal acetylation were set as variable modifications. Peptide spectrum matches (PSM) were filtered for Sequest HT Xcorr score ≥1. The Proteome Discoverer output file containing the LFQ abundances was loaded into Perseus (version 1.6.5.0) 71. Abundances were Log2-transformed and the proteins were filtered for at least two out of three valid values in one condition. Missing values were replaced by imputation based on the standard settings of Perseus, i.e. a normal distribution using a width of 0.3 and a downshift of 1.8. Differentially expressed proteins were determined using a t-test (threshold: FDR 1% or FDR 5% and S0: 0.13).

GENCODE annotations (gencode v19) were used to calculate number of amino acids per protein as well as the least distance between a particular amino-acid (W,Y and N) Boxplots for every group were plotted in R. Statistical tests were down using Wilcoxon test in R.

Proteome analysis for the detection of frameshifted polypeptides (relates to FIG. 4a-c)

I) Sample Preparation

For deeper proteome coverage in search of IFNγ-induced frameshifts, IFNγ- or mock treated melanoma cells were lysed and digested as described above, after which dried digests were subjected to basic reversed-phase (HpH-RP) high-performance liquid chromatography for offline peptide fractionation. 250 μg peptides were reconstituted in 95% 10 mM ammonium hydroxide (NH4OH, solvent A)/5% (90% acetonitrile (ACN)/10 mM NH4OH, solvent B) and loaded onto a Phenomenex Kinetex EVO C18 analytical column (150 mm×2.1 mm, particle size 5 μm, 100 Å pores) coupled to an Agilent 1260 HPLC system equipped with a fraction collector. Peptides were eluted at a constant flow of 100 μL/min in a 90-minute gradient containing a nonlinear increase from 5-30% solvent B. Fractions were collected and concatenated to 24 fractions per sample replicate. All fractions were analyzed by nanoLC-MS/MS on an Orbitrap Fusion Tribrid mass spectrometer equipped with an Easy-nLC1000 system (Thermo Scientific) as described previously⁷². Peptides were directly loaded onto the analytical column (ReproSil-Pur 120 C18-AQ, 1.9 μm, 75 μm×500 mm, packed in-house). Solvent A was 0.1% formic acid/water and solvent B was 0.1% formic acid/80% acetonitrile. Samples were eluted from the analytical column at a constant flow of 250 nl/min in a 2 h-gradient containing a linear increase from 8-32% solvent B.

II) In Silico Tryptophan-Associated Frameshift Database Construction

The CDS sequences of GRCh38 were downloaded from Ensembl⁷³. Prime transcripts (annotated as −001), which contain tryptophan codon and are highly expressed (log2(Normalized Read Counts) >5) in the ribosome profiling data were included for further analysis. Transcripts with less than 50 bp were discarded. Only CDS starting with ATG were kept. In cases of multiple in-frame TGG-codons per transcript, each TGG along the sequence was frameshifted separately. Both +1 and −1 frameshifts at the TGG codon position were implemented. The CDS out-of-frame was in silico translated until the first stop codon. Finally, we generated a database of chimeric polypeptides, starting at the first tryptic cleavage start site, upstream of tryptophan (33628 instances), frame-shifted at TGG codon (data not shown) via both −1 and +1 frame-shifts, until out-of-frame stop codons. No further filtering was implemented at the pre-scanning stage.

III) Data Search, Filtering and Analysis

Further, the MS data was analyzed by MaxQuant (version 1.6.0.16)74. The WT human proteome to run against was obtained from Uniprot database with SWISSPROT protein evidence level of 1⁷⁵. The in-frame protein expression data was further normalized and analyzed using DEP⁷⁶, For the frame-shift proteome analysis, the MS data was analyzed by MaxQuant (version 1.6.0.16)⁷⁴ with LFQ normalization, and then was scanned against the trans-frame polypeptide database together with the SWISSPROT proteins with Evidence level of 1. After scanning, a total of 124 peptides from the trans-frame polypeptide database were retained for further quantitative analysis, after subjecting to filtering for mapping to any proteomic and non-coding sequences ((data not shown)). Only the peptides reproducibly detected across replicates were retained for further analysis, and the list included reverse peptide hits (n=41) (FIG. 4c).

Immunopeptidomics analysis (relates to FIG. 4e)

I) Sample Preparation

Untreated MD55A3 (n=4), IFNγ treated (n=4), and Trp depleted (n=4) cell pellets were subjected to HLA-purification as described previously58,77, with slight modifications; Briefly, cell pellets were lysed with lysis buffer containing 0.25% sodium deoxycholate, 0.2 mM iodoacetamide, 1 mM EDTA, 1:200 protease inhibitors cocktail (Sigma-Aldrich), 1 mM PMSF and 1% octyl-b-D glucopyranoside in PBS, and then incubated at 4° C. for 1 hour. The lysates were cleared by centrifugation at 4° C. and 48,000g for 60 minutes, and then passed through a pre-clearing column containing Protein-A Sepharose beads (GenScript). HLA-I molecules were immunoaffinity purified from cleared lysate with the pan-HLA-I antibody (W6/32 antibody purified from HB95 hybridoma cells) covalently bound to Protein-A Sepharose beads). Affinity column was washed first with 10 column volumes of 400 mM NaCl, 20 mM Tris—HCl, pH 8.0 and then with 10 volumes of 20 mM Tris—HCl, pH 8.0. The HLA peptides and HLA molecules were eluted with 1% TFA followed by separation of the peptides from the proteins by binding the eluted fraction to disposable reversed-phase Sep-Pak tC18 (Waters). Elution of the peptides was done with 30% acetonitrile (ACN) in 0.1% trifluoracetic acid (TFA).

II) Liquid Chromatography MS-Analysis

The HLA peptides were dried by vacuum centrifugation, re-solubilized with formic acid and separated using reversed phase chromatography using the nanoAquity system (Waters Corp., USA), with a Symmetry trap column (180×20mm) and HSS T3 analytical column, 0.75×250mm (Waters Corp. USA). The chromatography system was coupled by electrospray to tandem mass spectrometry to Q-Exactive-Plus (Thermo Fisher Scientific). The HLA peptides were eluted with a linear gradient over 2 h from 5 to 28% acetonitrile with 0.1% formic acid at a flow rate of 0.35μl/min.

Data was acquired using a data-dependent “top 10” method, fragmenting the peptides by higher-energy collisional dissociation (HCD). Full scan MS spectra was acquired at a resolution of 70,000 at 200 m/z with a target value of 3×106 ions. Ions were accumulated to an AGC target value of 105 with a maximum injection time of generally 100 msec. The peptide match option was set to Preferred. Normalized collision energy was set to 25% and MS/MS resolution was 17,500 at 200 m/z. Fragmented m/z values were dynamically excluded from further selection for 20 sec.

Data Analysis

III) In-Silico Tryptophan-Associated Frameshift Database Construction

The CDS sequences of GRCh38 were downloaded from Ensembl⁷³. All transcript variants were included. Transcripts with less than 50 bp were discarded. Sequences in which no in-frame TGG codon (corresponding to tryptophan) exists were excluded. Only CDSs starting with ATG were kept. In cases where there were multiple in-frame TGG-codons per transcript, each TGG along the sequence was frameshifted separately. Both +1 and −1 frameshifts at the TGG codon position were implemented. The CDS out-of-frame was in silico translated until the first stop codon, or the next tryptophan obtained. The in-frame portion of the sequence was trimmed at the N′, such that it contained 12 amino acids upstream to the frameshift for the peptidomics database (as a 12 amino acid window upstream to the slippery tryptophan consists of all possible HLA I bound altered peptides, derived from this ribosomal slippage). At the last step, sequence redundancy was removed in cases of 100% sequence identity, and the longest sequence was kept using CD-HIT⁷⁸.

IV) Database Search and Filtration

The RAW MS data files were analyzed by MaxQuant (version 1.6.0.16). Files were searched against the frame-shifted database and the full canonical human proteome. The canonical human proteome was obtained from Ensembl GRCh38 and Uniprot database⁷⁵ following removal of 100% sequence redundancy using CD-HIT⁷⁸. The maximum allowed precursor mass tolerance was 20 ppm. N-terminal acetylation and methionine oxidation were set as variable modifications. A PSM false discovery rate (FDR) of 0.05 was used, and no protein FDR was set. Enzyme specificity was set as “unspecific” and “match between runs” option was set with default settings and LFQ was set to “minimum ratio count” of 1. The obtained peptides were filtered, by multiple criteria. Only peptides obtained by the frameshifted database and not the canonical database were kept. Peptides with Maxquant scores less than 80 or PEP larger than 0.1 were discarded. Any peptide not predicted by NetMHCpan (version 4.0)79 to bind the cell line HLA-alleles (either as strong or weak binders) were removed. In addition, in order to avoid false positive hits derived from poorly fragmented spectra and ambiguous sequence, we further filtered the detected peptides based on “Fragmentation coverage”(FC), defined as matched ions (b or y) divided by the total theoretical ions of the matched peptide sequence (peptide length-1). FC was calculated and only peptides showing MS/MS FC greater than 60% were kept. Peptides derived from source protein with expression in at least one dataset (transcriptome/translatome) were kept. Furthermore, peptides that were obtained in one or more control (non-treated) samples were not further investigated. Lastly, we confirmed that none of the corresponding identified aberrant peptides were generated from pseudogenes by aligning them to gencode protein-coding transcript sequences version³⁴, containing polymorphic pseudogenes entries. Similarly, we confirmed that the identified altered peptides were not derived from INDELS (using GATK4 version 4.1.4.1⁸⁰ haplotype caller for variant calling) or from intron retention (IR) events⁸¹.

Gibbs Clustering

Quality assessment of the identified peptides was done using the GibbsCluster2.0 server⁸² by clustering to 1-6 groups. These groups were compared to the expected motifs identified, using curated IEDB database⁸³.

V) Hydrophobicity Index (HI) Prediction

Sequence specific HI was calculated with an online available tool SSRCalc⁸⁴: available at hs2.proteome.ca/SSRCalc/SSRCalcQ.html. HI prediction obtained from the SSRCalc based on the 100Å C18 column, 0.1% formic acid separation system and without cysteine protection.

VI) Synthetic Peptide Validation

Light synthetic peptides for spectra validation were ordered from GenScript, as HPLC grade (≥85% purity). These were analyzed using the same LC-MSMS system and acquisition parameters as indicated above for the endogenous peptides, with the following changes: the gradient was from 4% to 30% acetonitrile in 20 min; NCE was set to 27. The data was processed with MaxQuant using the following parameters, all FDRs were set to 1, the individual peptide mass tolerance was set to false.

To compare the endogenous and synthetic spectra, we utilized the MSnbase R package 85 to calculate the Pearson correlations of fragment ions including a, b and y ions without neutral losses, detected in spectra of both endogenous and synthetic peptides, and to plot the head to tail graph. In all cases, we selected only the spectra that harbored the same precursor charge as the endogenous peptides and that were not post translationally modified. We then selected the synthetic spectra that had the best score in MaxQuant.

Selected peptides were ordered from JPT as synthetic peptides with one stable isotope-labeled amino acid, at >95% purity. The mass spectrometer was operated at a resolution of 70,000 (at m/z=200) for the MS1 full scan, scanning a mass range from 300 to 1650 m/z with an ion injection time of 120 ms and an AGC of 3e6. Then each peptide was isolated with an isolation window of 1.7 m/z prior to ion activation by high-energy collision dissociation (HCD, NCE=27). Targeted MS/MS spectra were acquired at a resolution of 35,000 (at m/z=200) with an ion injection time of 100 ms and an AGC of 2e⁵.

The PRM data were processed and analyzed by Skyline (v20.1.0.76)86, and an ion mass tolerance of 0.02 m/z was used to extract fragment ion chromatograms. Data was smoothed by the Savitzky Golay algorithm

Prediction of Disorderedness for Variant Peptides

The frame-shifted library for proteomics was subject to disorderedness prediction, albeit from the in-frame start codon. Only those peptides were retained for which in-frame part is longer than 25 amino-acids, while the out-of-frame part is longer than 30 amino-acids (stop codon occurs later). Disorderedness probability was obtained using IUPRED2A 87. Outlier groups were selected by using following cutoffs. Average out-of-frame (both -1 and +1) disordered score <0.15, difference between disorderedness prediction in-frame and out-of-frame part is less than 0.95 (FoldChange).

Cells and Reagents

Cell lines 12T and 108T were derived from pathology-confirmed metastatic melanoma tumor resections collected from patients enrolled in institutional review board (IRB)-approved clinical trials at the Surgery Branch of the National Cancer Institute. The MD55A3 cell line was derived from metastatic melanoma tumor resections collected with informed patient consent under a protocol approved by the NIH Institutional Review Board (IRB) Ethics Committee and approved by the MD Anderson IRB (protocol numbers 2012-0846, LABOO-063, and 2004-0069; NCT00338377). All cell lines were tested regularly and were found negative for mycoplasma contamination (EZ-PCR Mycoplasma Kit, Biological Industries). Cells were authenticated by Finger printing with STR profiling (Panel: PowerPlex_16_5Nov142UAGC, Size: GS500 ×35 ×50 ×250, Analysis Type: Fragment (Animal), Software Package: SoftGenetics GeneMarker 1.85). 12T, 108T, MD55A3, 888-Mel and D10 cells were cultured in Roswell Park Memorial Institute 1640 Medium (RPMI 1640, Gibco) supplemented with heat-inactivated 10% fetal bovine serum (Sigma), 25 mM HEPES (Gibco) and 100 U/mL penicillin/streptomycin (Gibco). HEK293T and A375 cells were cultured in Dulbecco's Modified Eagle's Medium (DMEM, Gibco), supplemented with 10% fetal bovine serum and 100 U/mL penicillin/streptomycin. All cell lines were maintained in a humidified atmosphere containing 5% of CO2 at 37° C. Tryptophan-free DMEM/F12 media was purchased from US Biologicals, custom-made tyrosine-free medium was custom purchased from Cell Culture Technologies, IFNγ (PeproTech) was used at 250 U/mL for the indicated durations of time. MG-132 (Selleckchem), dissolved in DMSO, was used at a final concentration of 10 μM. IDO inhibitor, 1-Methyl-L-tryptophan (Sigma) was dissolved in 0.1N NaOH at a 20 mM concentration, adjusted to pH=7.5, filter sterilized and used at a final concentration of 300 μM for 48 h. Polyethylenimine (PEI, Polysciences) was dissolved in water at a concentration of 1 mg/mL, after which it was filter sterilized, aliquoted and stored at −20° C.

Ribosome Profiling

The construction of ribosome protected fragments (RPF) libraries were done as previously described⁸⁸. For the generation of total RNA libraries, total RNA was extracted using TRI Reagent (Sigma), and mRNA was purified using Dynabeads mRNA DIRECT Purification Kit (Invitrogen), according to the manufacturers' protocol. Libraries were constructed using SENSE Total RNA-Seq Library Prep Kit for Illunima (Lexogen). RPF and Total RNA Libraries were loaded onto Illumina NextSeq 500 sequencer (Illumina)

Reporter Constructs

V5-ATF4-His and V5-ATF4-turboGFP reporter constructs were generated by PCR and restriction cloning into the XbaI-NotI sites of the lentiviral pCDH-Blasticidin vector (Addgene). V5-ATF4-His reporters were amplified using Phusion Polymerase (Thermo Fisher Scientific) using the primers listed below, with the pLX304-ORF clone of ATF4 (Dharmacon) as a template. V5-ATF4-tGFP reporters were amplified by a 2-step PCR, where first tGFP was amplified from a pLK0.1-tGFP vector (Addgene) with an overhang on the 5′ primer that matches the previously mentioned ATF4 reporters. In the 2nd PCR the V5-ATF4 part was added to it by using the V5-ATF4-His constructs as templates. The final constructs were sequence-verified by Sanger sequencing (Eurofins).

### Results

To address the long-term impact of sustained IFNγ-mediated tryptophan depletion on melanoma cells, we treated 12T melanoma cells with IFNγ for 48 hours. We confirmed the strong IDO1 induction and the consequential depletion of tryptophan, as well as the accumulation of kynurenine in IFNγ-treated cells (data not shown). We then performed ribosome profiling and analyzed the ribosome protected fragments (RPFs) by diricore (Differential Ribosome COdon REading) to detect differential ribosome occupancy patterns at the codon level²¹. We first examined the global distribution of RPFs along the transcripts. The metagene RPF distribution analysis of all expressed genes revealed an IFNγ-induced accumulation of RPFs at the translation start site of the transcripts, which corresponds to the expected reduction in global protein synthesis as measured by OP-Puro (O-propargyl-puromycin) incorporation assays (FIG. 1b and 1c). Subsequently, we investigated signals of shortage in specific amino acids by generating subsequence (codon occupancy bias) and 5′-RPF density plots 21 (data not shown). These analyses uncovered reduced ribosome occupancy at RPF position 12 (corresponding to reduced occupancy at the P-site of the ribosome) at the initiator methionine codon (ATGstart) (FIG. 1d, left panel), in line with global reduction in mRNA translation initiation. Similar results were obtained with the two additional melanoma cell lines MD55A3 and 108T (data not shown).

Further analysis of RPFs containing tryptophan codons at position 15 (A-site of the ribosome) revealed the impact of tryptophan shortage following IFNγ treatment on ribosome occupancy that indicated stalling of ribosomes on the tryptophan codon (FIG. 1e). 5′-RPF measurements confirmed the increase in the density of tryptophan codons at A-site of the ribosomes (FIG. 1d, middle panel, marked with arrow), indicative of ribosomal pausing at this site. In contrast, the codons of other amino acids showed no such pattern, suggesting a mechanism specifically directed by the lack of tryptophan (e.g. TGT Cys codon, 1d. right panel).

Similar results were obtained in two other melanoma cell lines (data not shown). These observations are consistent with the expected suppressed translation initiation and stalling of ribosomes at the Trp codon due to tryptophan shortage²¹.

Surprisingly, in all the examined melanoma cell lines, we observed a massive accumulation of RPFs downstream of tryptophan codons, which we hereafter refer to as W-Bumps (FIG. 1d). We verified the presence of W-Bumps on individual genes. Figure if shows an enhanced accumulation in RPFs following the tryptophan codons of two genes (ATF4 and CDC6, grey background) as opposed to the tryptophan-less ATP5G1 gene. The existence of W-Bumps downstream of tryptophan codons suggests an unexpected and unidentified bypass of ribosomes of these sites in IFNγ-treated cells.

Next, we performed a set of experiments to better characterize the occurrence of W-Bumps. We first examined IDO1-dependency by chemically inhibiting IDO1 activity using the IDO inhibitor 1-Methyl-L-tryptophan (IDOi). IDOi addition to IFNγ-treated melanoma cells strongly inhibited IDO1's enzymatic activity without affecting its transcriptional induction. As expected, IDOi negated the global redistribution of RPFs towards the translation initiation sites and the enrichment of the tryptophan codon signal at A-site (FIGS. 1g-i). More importantly, IDOi also rescued the accumulation of RPFs at the W-Bumps region (FIG. 1h middle panel). To further substantiate the involvement of tryptophan shortage in W-Bumps generation, we depleted tryptophan and performed ribosome profiling and subsequent diricore analysis. As expected, tryptophan depletion phenocopied IFNγ treatment (data not shown). It inhibited global initiation of protein synthesis, reduced RPF density at the initiator ATG P-site, and most importantly also generated W-bumps (data not shown). Altogether, the exhaustion of tryptophan not only causes ribosome stalling at tryptophan codons but also induces vast accumulation downstream thereof.

To further characterize W-Bumps in an unbiased manner, we constructed a bioinformatics pipeline (Bump-finder) to unbiasedly identify regional accumulations of RPFs in ribosome profiling data (FIG. 2a). The vicinity of detected bump regions was then scanned for the frequencies of codons for each individual amino acid. Interestingly, while none of the codons showed any particular enrichment in the vicinity of bumps in control cells, IFNγ treatment increased a tryptophan signal at a location —20 amino acids upstream to the peak of the detected bumps (FIG. 2b). We further substantiated the link between IFNγ-induced bumps and tryptophan by examining the ratio of the abundance of each codon at a distance of 30 triplets upstream and downstream of the detected bumps. In all three melanoma cell lines examined, IFNγ treatment induced enrichment of tryptophan codons upstream of the identified bump signals (FIG. 2c). To investigate this in detail, we analyzed the occurrence of bumps right after each tryptophan codon. While in control cells no bump signal appeared, IFNγ-treatment induced bumps downstream of tryptophan codons (FIG. 2d). A similar, but scaled-up analysis focused on all tryptophans, indicated that tryptophan-associated bumps are a widespread phenomenon upon IFNγ-treatment (FIG. 2e). The analysis of IDOi and tryptophan depletion datasets further strengthened the connection of bumps to tryptophan. Bumps were completely abolished by IDOi treatment, and recapitulated by tryptophan depletion (FIG. 2f and g). To assess whether the induction of bumps is a global phenomenon that relates to amino acid shortages, we performed diricore analysis of ribosome profiling datasets of 12T melanoma cells deprived of tyrosine. Interestingly, under these conditions we identified the induction of Y-Bumps, which featured similar characteristics as W-Bumps, but instead were only associated with tyrosine (FIG. 2h).

Next, we attempted to identify coding elements associated with W-Bump formation. We selected tryptophan codons that were either strongly or weakly associated with W-Bumps (FIG. 2i), and searched for surrounding mRNA and codon signatures enriched in the group of IFNγ-induced W-Bumps. The only delineating signal associated with strong W-Bumps was the presence of multiple in frame tryptophan codons within a region of 8 codons (FIGS. 2j and 2k).

To look at the downstream consequences of W-Bump formation, we assessed the impact of W-Bumps on protein expression by proteomic analysis following IFNγ induction. We first analyzed the differential protein expression in IFNγ-treated versus control cells (data not shown) and grouped proteins according to the number of tryptophan residues they contain (FIG. 2l). This analysis indicated an association between the number of tryptophan residues in a given protein and a reduction in the protein level in response to IFNγ (FIG. 2l). Interestingly, no such association was observed in a similar analysis for mRNA levels, indicating a post-transcriptional effect (FIG. 2l). Importantly, this association was specific to tryptophan residues (FIG. 2m). This result could be either explained by an increased degradation (as suggested by Vabulas and Hartl²²), or by a lowered translation rate of proteins containing tryptophans. To distinguish between these possibilities, we determined protein levels in response to a combined treatment of cells with IFNγ and a potent proteasome inhibitor, MG132 (data not shown). This treatment did not restore the reduced levels of the high-tryptophan containing proteins (FIG. 2n), suggesting that IFNγ stimulus specifically affects the translation of tryptophan-containing proteins. As noted above, control amino acids showed no such effect (FIG. 2o). Further analysis excluded protein length as a contributing factor (data not shown).

To link W-Bumps to the detected decrease in protein synthesis, we analyzed two groups of proteins, one group with two tryptophans encoded within a stretch of eight amino acids (named <8), the other with two tryptophans separated by more than eight amino acids (named >8). Interestingly, the <8 group showed a stronger W-Bumps signal compared to >8 (FIG. 2p, 2q). In addition, the proteomics data revealed a greater IFNγ-induced reduction in the expression of proteins in the <8 group compared to the >8 group (p-value 2×10−6, FIG. 2r). In contrast, a similar analysis of a control residue did not show a significant effect (FIG. 2s). Altogether, our results pinpoint the biological importance of W-Bumps in restraining protein synthesis upon IFNγ signaling.

Given the average distance of approximately 20 codons between the W-Bumps and the tryptophan codon, and the periodicity of tryptophan codons within the Bumps, we hypothesized that W-Bumps might be connected to the secondary structure of the nascent peptide in the ribosomal exit tunnel. Though little is known about the influence of secondary structure in this exit tunnel on ribosomal stalling, the formation of an α-helical secondary structure in the tunnel zone is thought to be a major determinant for ribosomal progression²³⁻²⁸. Indeed, peptide sequences corresponding to the W-Bumps region form an a-helix structure more frequently than other regions in the proteome (FIG. 3a). Possibly, the loss of this α-helical structure in peptide sequences that are normally rich in tryptophan could induce stalling of ribosomes. One interesting possibility leading to loss of this α-helical peptide sequence would be frameshifting events, which are known to occur at sites of ribosome stalling after amino acid starvations (FIG. 3b)²⁹⁻³⁸. To examine this in-silico, we scored for disorderedness of in-frame and out-of-frame peptides by computationally introducing frameshifts at the site of all tryptophan codons. In general, the level of disorderedness of the newly formed peptide after the frameshift greatly increases, supporting our hypothesis (FIG. 3c, green line). Remarkably, a selected outlier group showed highly ordered out-of-frame peptides downstream of the tryptophan (FIG. 3c, red line). While in general, the out-of-frame regions downstream of tryptophan are associated with W-Bumps, the selected group with ordered regions are not (FIG. 3d). Therefore, W-Bumps could at least in part be the result of ribosomes that bypassed tryptophan codons by frameshifting events, but then paused with out-of-frame aberrant polypeptides in their lower exit tunnel (FIG. 3b).

To experimentally confirm the occurrence of frameshifting events, we used V5-ATF4(1-63)-His lentiviral reporter constructs containing the first 63 amino acids of ATF4 (with a tryptophan residue at position 60, preceding a W-Bump, FIG. 1f) flanked with V5- and His-tags at the N- and C-termini, respectively. We generated one intact (with the His-tag in-frame) and two constructs with the His-tag out-of-frame (+1, +2, FIG. 3e). Only upon frameshifting events surrounding the tryptophan codon would the His-tag be expressed from the +1 and +2 constructs, whereby it can be pulled down (FIG. 3f). We stably expressed the three constructs in MD55A3 melanoma cells and examined reporter expression in either mock or IFNγ-treated cells using His-tag pulldowns, followed by V5-tag immunoblotting. FIG. 3g shows that the in-frame reporter protein was very efficiently pulled down through its His-tag in both control and IFNγ treated conditions. In contrast, both the +1 and +2 reporter proteins were retained in the supernatant in control conditions but partially pulled down following IFNγ treatment, indicative of frameshifting events (FIG. 3g). Interestingly, when the supernatant of lysates containing the in-frame reporter was subjected to anti-V5 immunoprecipitation to enrich for residual V5-tagged proteins lacking a His-tag at their C-terminus, only in the IFNγ-induced condition such proteins were detected (data not shown), supporting the production of trans-frame products also in the in-frame reporter situation.

When we included an IDOi treatment, IFNγ-induced frameshifting was prevented, confirming the causative role of tryptophan shortage for frameshifts to occur (FIG. 3h). In addition, frameshifting events were clearly present under tryptophan depleted conditions (FIG. 3i). To confirm the importance of the tryptophan codon in IFNγ-induced frameshifting, we substituted it with a tyrosine codon, and observed cessation of frameshifting in both out-of-frame reporters (FIG. 3j). Instead, the depletion of tyrosine (which is associated with Y-Bumps formation (FIG. 2h)) from cells containing the tryptophan to tyrosine mutated reporter, now led to frameshifting (data not shown). Lastly, in order to confirm the IFNγ-induced frameshifting with a different set of reporter constructs, and to evaluate the cell population percentage that undergoes IFNγ-induced ribosomal frameshifting, we substituted the His-tag for a turboGFP (tGFP) gene. Comprising a considerably larger polypeptide sequence than the His-tag, tGFP contains no tryptophan residues and allows for fluorescence readout. Similar to the V5-ATF(1-63)-His tagged protein, also the V5-ATF4(1-63)-tGFP vector showed the occurrence of frameshifts by an increase in fluorescent signal of +1 and +2 constructs following IFNγ treatment, while the signal of the in-frame construct remained unchanged (FIG. 3k). The production of the trans-frame tGFP products was validated using anti-V5 and anti-tGFP immunoblotting analyses (FIG. 31).

Next, we examined the production of trans-frame products in the context of a T-cell attack, where IFNγ is locally secreted upon recognition of an antigen on the target cells. We co-cultured anti-melan A (MART-1) T-cells³⁹ with either IFNγ-sensitive (D10) or the more resistant (888-Mel) melanoma cells, both expressing the MART-1 antigen and the V5-ATF4(1-63)-His tagged reporter gene either in- or out-of-frame (Frame and +1, respectively). V5-tag immunoblot analysis of His-tag pulldowns demonstrated that frameshifting events occur also in this native context (FIG. 3m). Interestingly, frameshifting was only apparent in the sensitive D10 cells, in line with the magnitude of IDO1 protein induction in these cells (Extended Data FIG. 4g). The effect of the T-cell interaction was recapitulated by IFNγ treatment, showing frameshifting events in D10 cells, coinciding with a much stronger IDO1 upregulation and a larger tryptophan depletion in these cells when compared with 888-Mel (data not shown). In contrast to IFNγ treatment, tryptophan depletion induced frameshifting events to a similar extent in both cell lines (FIG. 3n), indicating that the weaker IDO1 induction in 888-Mel by IFNγ signaling is the likely cause of the lower frameshifting rate. Altogether, this confirms the causal role of tryptophan depletion in the induction of trans-frame protein production.

We next aimed to search for aberrant peptides in the full proteome using an in silico generated database of −1 and +1 frameshifts introduced specifically at the sites of Trp codons. IFNγ-treated and control MD55A3 cells expressing the out-of-frame tGFP+1reporter gene were subjected to 2D-LC MS/MS. A PCA plot analysis indicated overall good reproducibility of this data (data not shown). Differential expression analysis confirmed upregulation of IFNγ signature (FIG. 4a). Next, we searched for peptides corresponding to our tGFP reporter, and found that IFNγ treatment markedly induced its expression (>30-fold), demonstrating the feasibility of detecting frameshifted peptides (FIG. 4b). To identify Trp-associated trans-frame proteins, we predicted out-of-frame −1 and +1 polypeptides created by frameshifting at endogenous tryptophans of proteins expressed in MD55A3 cells, as determined by ribosome profiling (resulting in 66728 trans-frame polypeptides created from 33624 tryptophans). This library was supplemented with the database containing the entire proteome and used for scanning of the 2D-LC MS/MS data. This analysis led to the detection of 124 out-of-frame and trans-frame peptides not present in any of the in-frame polypeptides (including pseudogenes, alternative mRNA isoforms or upstream open reading frames (data not shown), 41 of them were reproduced in two biological replicates (FIG. 4c). Remarkably, whereas IFNγ treatment led to reduced intensity of peptides from the proteomic source (data not shown), likely due to its effect on mRNA translation (FIG. 1c), a significantly higher number of frameshifted peptides was induced in this condition (34 out of 41, p=4.42e⁻¹⁰, FIG. 4c). This induction was not observed in the corresponding in-frame genes of the frameshifted peptides (marked “in frame” FIG. 4c). We controlled this result by analyzing the sequences of the out-of-frame polypeptide library in a reverse orientation, yielding only 3 peptides, none of which were induced by IFNγ (FIG. 4c). This data demonstrates the occurrence of endogenous frameshifting events at tryptophan residues following IFNγ treatment.

Notably, in addition to the strong IFNγ protein signature in the IFNγ-treated cells (FIG. 4a), a strong induction of immunoproteasome subunits and an upregulation of antigen presentation via HLA molecules was observed^{40,41} (data not shown). Since it was reported that a significant source of the HLA-presented peptides are derived from newly synthesized, rapidly degraded proteins⁴²⁻⁴⁴ as well as from cryptic, non-canonical ORF translation⁴⁵⁻⁵⁴, we asked whether the translationally altered polypeptides that are generated as a result of IFNγ-induced Trp depletion, may be a source for peptide presentation on MHC molecules. To investigate this possibility, we tested the efficiency of presentation of frameshifted peptides using the model peptide SIINFEKL from mouse ovalbumin that binds to H2-Kb⁵⁵. A375 melanoma cells, which have the capacity to frameshift (data not shown), were engineered to express H2-Kb and either our in-frame or +1 out-of-frame tGFP reporters that were extended with the SIINFEKL peptide sequence. FIG. 4d shows that the in-frame SIINFEKL peptide is well expressed and presented, and its presentation is mildly induced by IFNγ treatment in an IDO1-independent manner. This IFNγ-mediated induction of SIINFEKL presentation can be explained by an enhanced antigen processing machinery (data not shown). In contrast, the presentation of the +1 out-of-frame SIINFEKL peptide was essentially undetectable in mock treated cells, and was strongly stimulated upon IFNγ treatment in an IDO1-dependent manner (FIG. 4d). These results indicate that the IDO1-mediated tryptophan depletion, following IFNγ stimulus, results in ribosomal frameshifting events that can lead to aberrant peptide presentation.

Next, we examined the presentation of aberrant endogenous peptides using an immuno-peptidomics analysis⁵⁸ of MD55A3 cells, either untreated or treated with IFNγ, or grown in tryptophan-free media for 48 h (data not shown). To detect W-associated out-of-frame peptides, we generated a database containing −1 and +1 polypeptides sequences starting 12 aa before tryptophans until the nearest stop codon, or the next Trp codon. We then searched for these translationally altered peptides in immunopeptidomics data derived from MD55A3 cells as well as in immunopeptidomics data from fresh metastases derived from the same patient (patient 55 58), following careful, sequential filtration steps. Interestingly, we detected 94 HLA I-bound aberrant peptides, containing both −1 and +1 out-of-frame and chimeric, trans-frame sequences. Thirteen of those were found presented exclusively in the fresh metastatic samples, and 81 were found in the different MD55A3 cell samples. Remarkably, a comparison between the relative intensities of the altered peptides presented in the treated versus untreated cells, revealed an enrichment in the treated cells (data not shown). Importantly, out of the fifteen aberrant peptides that were found presented exclusively in the treated samples (either IFNγ, Trp depletion, or both), six were also detected in the metastases (data not shown).

Overall, the identified altered peptides found in the treated MD55A3 cells and in the fresh metastases (n=28) shared similar properties with the in-frame HLA-I bound peptides (data not shown). In order to validate the identification of those peptides, we generated synthetic peptides, analyzed them, and compared their resulting MS/MS spectra with those of the native, endogenous aberrant peptides. This derived 20 peptides that have a good correlation between their native and synthetic counterparts FIG. 4e). In addition, two peptides derived from −1 and +1 frameshifts were further validated by spiking stable isotopically labeled peptides that co-eluted with them (data not shown).

Interestingly, analysis of a metastatic sample derived from another patient (patient 42⁵⁸), who shares three of six HLA class I alleles with patient 55 (A03:01; B07:02; C07:02), revealed that three of the identified peptides were identical (data not shown), suggesting that some of the identified altered peptides may be recurrent across patients. These results confirmed the endogenous production of aberrant peptides and their presentation on the cell surface.

We next asked whether the HLA complexes containing these altered peptides can be immunogenic. Previous studies have shown that T cells from healthy donors, unbiased by the immunosuppressive mechanisms occurring in patients, can recognize tumor-specific peptides ignored by tumor-infiltrating T cells⁵⁹. The reactive donor-derived T cells are confined to the naive T cell compartment⁶⁰. Aberrant peptides identified in our immuno-peptidomic analysis (list of tested peptides outlined in Methods section) presented on relevant HLA were hence tested for ability to prime naive CD8+ T cells from healthy donors, as previously described⁶⁰. Briefly, monocyte-derived dendritic cells (MoDCs) isolated from healthy donor peripheral blood mononuclear cells (PBMCs) were peptide-pulsed with aberrant peptides and co-cultured with autologous naive CD8+ T cells. Following 10 days of co-culture, combinatorial tetramer staining analysed by flow cytometry revealed CD8+ T cells reactive to two aberrant peptides in two different donors; an HLA-B*07:02-restricted KCNK6-derived aberrant peptide and an HLA-C*07:02-restricted ZNF513-derived aberrant peptide (FIG. 4f). Reactive T cells were identified as live CD8+ T cells staining positively in two pMHC multimer channels (for increased specificity) and negatively in three other pMHC multimer channels (data not shown). T cells staining positively with pMHC-multimers were sorted as single cells to generate T-cell clones. Sixteen T cell clones reactive to the KCNK6-derived aberrant peptide, predicted to bind strongly to HLA-B*07:02 (NetMHCpan 4.1), expanded sufficiently for subsequent analysis. Thirteen out of sixteen KCNK6 clones stained positively with the relevant multimers and were strongly activated by HLA-B*07:02pos target cells loaded with relevant peptide (FIG. 4g). Sorted T cells reactive to the ZNF513-derived aberrant peptide did not expand sufficiently for further analysis. These data demonstrate that aberrant peptides derived as a consequence of IFNγ exposure can be presented on HLA molecules to T cells and induce an immune response.

In summary, we show that melanoma cells exposed to prolonged IFNγ exposure derived from their interaction with T cells, induce an IDO1-mediated tryptophan depletion. Despite this depletion and despite the stalling of ribosomes on tryptophan codons, mRNA translation proceeds via ribosomal frameshifting. This leads on the one hand to ribosome stalling downstream of the tryptophan codon due to out-of-frame and trans-frame aberrant peptide production and loss of secondary structure within the ribosome exit tunnel. Most notably, these aberrant peptides can be detected in the full proteome, can be found presented on HLA class I molecules on melanoma cells, and can prime T cells. Our findings of this novel translational mechanism by which cancer cells cope with amino acid shortages are of particular relevance as they provide a new layer to the complex landscape of melanoma-presented HLA-peptides.

### REFERENCES

1 Ayers, M. et al. IFN-gamma-related mRNA profile predicts clinical response to PD-1 blockade. J Clin Invest 127, 2930-2940, doi:10.1172/JCI91190 (2017).
2 Gajewski, T. F. et al. Cancer Immunotherapy Targets Based on Understanding the T Cell-Inflamed Versus Non-T Cell-Inflamed Tumor Microenvironment. Adv Exp Med Biol 1036, 19-31, doi:10.1007/978-3-319-67577-0_2 (2017).
3 Ji, R. R. et al. An immune-active tumor microenvironment favors clinical response to ipilimumab. Cancer Immunol Immunother 61, 1019-1031, doi:10.1007/s00262-011-1172-6 (2012).
4 Platten, M., Wick, W. & Van den Eynde, B. J. Tryptophan catabolism in cancer: beyond IDO and tryptophan depletion. Cancer Res 72, 5435-5440, doi:10.1158/0008-5472.CAN-12-0569 (2012).
5 Timosenko, E. et al. Nutritional Stress Induced by Tryptophan-Degrading Enzymes Results in ATF4-Dependent Reprogramming of the Amino Acid Transporter Profile in Tumor Cells. Cancer Res 76, 6193-6204, doi:10.1158/0008-5472.CAN-15-3502 (2016).
6 Zhai, L. et al. Molecular Pathways: Targeting IDO1 and Other Tryptophan Dioxygenases for Cancer Immunotherapy. Clin Cancer Res 21, 5427-5433, doi:10.1158/1078-0432.CCR-15-0420 (2015).
7 Garcia-Jimenez, C. & Goding, C. R. Starvation and Pseudo-Starvation as Drivers of Cancer Metastasis through Translation Reprogramming Cell Metab 29, 254-267, doi:10.1016/j.cmet.2018.11.018 (2019).
8 Amobi, A., Qian, F., Lugade, A. A. & Odunsi, K. Tryptophan Catabolism and Cancer Immunotherapy Targeting IDO Mediated Immune Suppression. Adv Exp Med Biol 1036, 129-144, doi:10.1007/978-3-319-67577-0_9 (2017).
9 Labadie, B. W., Bao, R. & Luke, J. J. Reimagining IDO Pathway Inhibition in Cancer Immunotherapy via Downstream Focus on the Tryptophan-Kynurenine-Aryl Hydrocarbon Axis. Clin Cancer Res 25, 1462-1471, doi:10.1158/1078-0432.CCR-18-2882 (2019).
10 Komiya, T. & Huang, C. H. Updates in the Clinical Development of Epacadostat and Other Indoleamine 2,3-Dioxygenase 1 Inhibitors (IDO1) for Human Cancers. Front Oncol 8, 423, doi:10.3389/fonc.2018.00423 (2018).
11 Gunther, J., Dabritz, J. & Wirthgen, E. Limitations and Off-Target Effects of Tryptophan-Related IDO Inhibitors in Cancer Treatment. Front Immunol 10, 1801, doi:10.3389/fimmu.2019.01801 (2019).
12 Opitz, C. A. et al. The therapeutic potential of targeting tryptophan catabolism in cancer. Br J Cancer, doi:10.1038/s41416-019-0664-6 (2019).
13 Battu, S., Minhas, G., Mishra, A. & Khan, N. Amino Acid Sensing via General Control Nonderepressible-2 Kinase and Immunological Programming Front Immunol 8, 1719, doi:10.3389/fimmu.2017.01719 (2017).
14 Wek, R. C. & Staschke, K. A. How do tumours adapt to nutrient stress? EMBO J 29, 1946-1947, doi:10.1038/emboj.2010.110 (2010).
15 Ye, J. et al. The GCN2-ATF4 pathway is critical for tumour cell survival and proliferation in response to nutrient deprivation. EMBO J 29, 2082-2096, doi:10.1038/emboj.2010.81 (2010).
16 Harding, H. P. et al. Regulated translation initiation controls stress-induced gene expression in mammalian cells. Mol Cell 6, 1099-1108, doi:10.1016/s1097-2765(00)00108-8 (2000).
17 Falletta, P. et al. Translation reprogramming is an evolutionarily conserved driver of phenotypic plasticity and therapeutic resistance in melanoma. Genes Dev 31, 18-33, doi:10.1101/gad.290940.116 (2017).
18 Darnell, A. M., Subramaniam, A. R. & O'Shea, E. K. Translational Control through Differential Ribosome Pausing during Amino Acid Limitation in Mammalian Cells. Mol Cell 71, 229-243 e211, doi:10.1016/j.molcel.2018.06.041 (2018).
19 Wu, C. C., Peterson, A., Zinshteyn, B., Regot, S. & Green, R. Ribosome Collisions Trigger General Stress Responses to Regulate Cell Fate. Cell, doi:10.1016/j.cell.2020.06.006 (2020).
20 Adam, I. et al. Upregulation of tryptophanyl-tRNA synthethase adapts human cancer cells to nutritional stress caused by tryptophan degradation. Oncoimmunology 7, e1486353, doi:10.1080/2162402X.2018.1486353 (2018).
21 Loayza-Puch, F. et al. Tumour-specific proline vulnerability uncovered by differential ribosome codon reading. Nature 530, 490-494, doi:10.1038/nature16982 (2016).
22 Vabulas, R. M. & Hartl, F. U. Protein synthesis upon acute nutrient restriction relies on proteasome function. Science 310, 1960-1963, doi:10.1126/science.1121925 (2005).
23 Mingarro, I., Nilsson, I., Whitley, P. & von Heijne, G. Different conformations of nascent polypeptides during translocation across the ER membrane. BMC Cell Biol 1, 3, doi:10.1186/1471-2121-1-3 (2000).
24 Bhushan, S. et al. alpha-Helical nascent polypeptide chains visualized within distinct regions of the ribosomal exit tunnel. Nat Struct Mol Biol 17, 313-317, doi:10.1038/nsmb.1756 (2010).
25 Woolhead, C. A., McCormick, P. J. & Johnson, A. E. Nascent membrane and secretory proteins differ in FRET-detected folding far inside the ribosome and in their exposure to ribosomal proteins. Cell 116, 725-736, doi:10.1016/s0092-8674(04)00169-2 (2004).
26 Lu, J. & Deutsch, C. Secondary structure formation of a transmembrane segment in Kv channels. Biochemistry 44, 8230-8243, doi:10.1021/bi050372q (2005).
27 Tu, L. & Deutsch, C. Determinants of Helix Formation for a Kv1.3 Transmembrane Segment inside the Ribosome Exit Tunnel. J Mol Biol 429, 1722-1732, doi:10.1016/j.jmb.2017.04.022 (2017).
28 Bano-Polo, M. et al. Transmembrane but not soluble helices fold inside the ribosome tunnel. Nat Commun 9, 5246, doi:10.1038/s41467-018-07554-7 (2018).
29 Caliskan, N. et al. Conditional Switch between Frameshifting Regimes upon Translation of dnaX mRNA. Mol Cell 66, 558-567 e554, doi:10.1016/j.molcel.2017.04.023 (2017).
30 Yelverton, E., Lindsley, D., Yamauchi, P. & Gallant, J. A. The function of a ribosomal frameshifting signal from human immunodeficiency virus-1 in Escherichia coli. Mol Microbiol 11, 303-313, doi:10.1111/j.1365-2958.1994.tb00310.x (1994).
31 Atkinson, J., Dodge, M. & Gallant, J. Secondary structures and starvation-induced frameshifting. Mol Microbiol 26, 747-753, doi:10.1046/j.1365-2958.1997.6101959.x (1997).
32 Lindsley, D. & Gallant, J. On the directional specificity of ribosome frameshifting at a “hungry” codon. Proc Natl Acad Sci U S A 90, 5469-5473, doi:10.1073/pnas.90.12.5469 (1993).
33 Gurvich, O. L., Baranov, P. V., Gesteland, R. F. & Atkins, J. F. Expression levels influence ribosomal frameshifting at the tandem rare arginine codons AGG_AGG and AGA_AGA in Escherichia coli. J Bacteriol 187, 4023-4032, doi:10.1128/JB.187.12.4023-4032.2005 (2005).
34 Olubajo, B. & Taylor, E. W. A −1 frameshift in the HIV-1 env gene is enhanced by arginine deficiency via a hungry codon mechanism. Mutat Res 579, 125-132, doi:10.1016/j.mrfmmm.2005.02.018 (2005).
35 Barak, Z., Lindsley, D. & Gallant, J. On the mechanism of leftward frameshifting at several hungry codons. J Mol Biol 256, 676-684, doi:10.1006/jmbi.1996.0117 (1996).
36 Laine, S., Thouard, A., Komar, A. A. & Rossignol, J. M. Ribosome can resume the translation in both +1 or −1 frames after encountering an AGA cluster in Escherichia coli. Gene 412, 95-101, doi:10.1016/j.gene.2008.01.018 (2008).
37 Spanjaard, R. A., Chen, K., Walker, J. R. & van Duin, J. Frameshift suppression at tandem AGA and AGG codons by cloned tRNA genes: assigning a codon to argU tRNA and T4 tRNA(Arg). Nucleic Acids Res 18, 5031-5036, doi:10.1093/nar/18.17.5031 (1990).
38 Temperley, R., Richter, R., Dennerlein, S., Lightowlers, R. N. & Chrzanowska-Lightowlers, Z. M. Hungry codons promote frameshifting in human mitochondrial ribosomes. Science 327, 301, doi:10.1126/science.1180674 (2010).
39 Vredevoogd, D. W. et al. Augmenting Immunotherapy Impact by Lowering Tumor TNF Cytotoxicity Threshold. Cell 178, 585-599 e515, doi:10.1016/j.cell.2019.06.014 (2019).
40 Zhou, F. Molecular mechanisms of IFN-gamma to up-regulate MHC class I antigen processing and presentation. Int Rev Immunol 28, 239-260, doi:10.1080/08830180902978120 (2009).
41 Vigneron, N. & Van den Eynde, B. J. Proteasome subtypes and the processing of tumor antigens: increasing antigenic diversity. Curr Opin Immunol 24, 84-91, doi:10.1016/j.coi.2011.12.002 (2012).
42 Bourdetsky, D., Schmelzer, C. E. & Admon, A. The nature and extent of contributions by defective ribosome products to the HLA peptidome. Proc Natl Acad Sci U S A 111, E1591-1599, doi:10.1073/pnas.1321902111 (2014).
43 Yewdell, J. W. DRiPs solidify: progress in understanding endogenous MHC class I antigen processing. Trends Immunol 32, 548-558, doi:10.1016/j.it.2011.08.001 (2011).
44 Trentini, D. B. et al. Role for ribosome-associated quality control in sampling proteins for MHC class I-mediated antigen presentation. Proc Natl Acad Sci U S A 117, 4099-4108, doi:10.1073/pnas.1914401117 (2020).
45 Erhard, F. et al. Improved Ribo-seq enables identification of cryptic translation events. Nat Methods 15, 363-366, doi:10.1038/nmeth.4631 (2018).
46 Prasad, S., Starck, S. R. & Shastri, N. Presentation of Cryptic Peptides by MHC Class I Is Enhanced by Inflammatory Stimuli. J Immunol 197, 2981-2991, doi:10.4049/jimmunol.1502045 (2016).
47 Starck, S. R. & Shastri, N. Nowhere to hide: unconventional translation yields cryptic peptides for immune surveillance. Immunol Rev 272, 8-16, doi:10.1111/imr.12434 (2016).
48 Chen, J. et al. Pervasive functional translation of noncanonical human open reading frames. Science 367, 1140-1146, doi:10.1126/science.aay0262 (2020).
49 Chong, C. et al. Integrated proteogenomic deep sequencing and analytics accurately identify non-canonical peptides in tumor immunopeptidomes. Nat Commun 11, 1293, doi:10.1038/s41467-020-14968-9 (2020).
50 Laumont, C. M. & Perreault, C. Exploiting non-canonical translation to identify new targets for T cell-based cancer immunotherapy. Cell Mol Life Sci 75, 607-621, doi:10.1007/s00018-017-2628-4 (2018).
51 Laumont, C. M. et al. Noncoding regions are the main source of targetable tumor-specific antigens. Sci Transl Med 10, doi:10.1126/scitranslmed.aau5516 (2018).
52 Pearson, H. et al. MHC class I-associated peptides derive from selective regions of the human genome. J Clin Invest 126, 4690-4701, doi:10.1172/JCI88590 (2016).
53 Laumont, C. M. et al. Global proteogenomic analysis of human MHC class I-associated peptides derived from non-canonical reading frames. Nat Commun 7, 10238, doi:10.1038/ncomms10238 (2016).
54 Saulquin, X. et al. +1 Frameshifting as a novel mechanism to generate a cryptic cytotoxic T lymphocyte epitope derived from human interleukin 10. J Exp Med 195, 353-358, doi:10.1084/jem.20011399 (2002).
55 Dersh, D., Yewdell, J. W. & Wei, J. A SIINFEKL-Based System to Measure MHC Class I Antigen Presentation Efficiency and Kinetics. Methods Mol Biol 1988, 109-122, doi:10.1007/978-1-4939-9450-2_9 (2019).
56 McCarthy, M. K. & Weinberg, J. B. The immunoproteasome and viral infection: a complex regulator of inflammation. Front Microbiol 6, 21, doi:10.3389/fmicb.2015.00021 (2015).
57 Goldberg, A. L., Cascio, P., Saric, T. & Rock, K. L. The importance of the proteasome and subsequent proteolytic steps in the generation of antigenic peptides. Mol Immunol 39, 147-164, doi:10.1016/s0161-5890(02)00098-6 (2002).
58 Kalaora, S. et al. Combined Analysis of Antigen Presentation and T-cell Recognition Reveals Restricted Immune Responses in Melanoma. Cancer Discov 8, 1366-1375, doi:10.1158/2159-8290.CD-17-1418 (2018).
59 Stronen, E. et al. Targeting of cancer neoantigens with donor-derived T cell receptor repertoires. Science 352, 1337-1341, doi:10.1126/science.aaf2288 (2016).
60 Ali, M. et al. Induction of neoantigen-reactive T cells from healthy donors. Nat Protoc 14, 1926-1943, doi:10.1038/s41596-019-0170-6 (2019).
61 Frankish, A. et al. GENCODE reference annotation for the human and mouse genomes. Nucleic Acids Res 47, D766-D773, doi:10.1093/nar/gky955 (2019).
62 Langmead, B. Aligning short sequencing reads with Bowtie. Curr Protoc Bioinformatics Chapter 11, Unit 11 17, doi:10.1002/0471250953.bi1107s32 (2010).
63 Kim, D. et al. TopHat2: accurate alignment of transcriptomes in the presence of insertions, deletions and gene fusions. Genome Biol 14, R36, doi:10.1186/gb-2013-14-4-r36 (2013).
64 Lauria, F. et al. riboWaltz: Optimization of ribosome P-site positioning in ribosome profiling data. PLoS Comput Biol 14, e1006169, doi:10.1371/journal.pcbi.1006169 (2018).
65 Li, H. et al. The Sequence Alignment/Map format and SAMtools. Bioinformatics 25, 2078-2079, doi:10.1093/bioinformatics/btp352 (2009).
66 Anders, S., Pyl, P. T. & Huber, W. HTSeq—a Python framework to work with high-throughput sequencing data. Bioinformatics 31, 166-169, doi:10.1093/bioinformatics/btu638 (2015).
67 Anders, S. & Huber, W. Differential expression analysis for sequence count data. Genome Biol 11, R106, doi:10.1186/gb-2010-11-10-r106 (2010).
68 Quinlan, A. R. & Hall, I. M. BEDTools: a flexible suite of utilities for comparing genomic features. Bioinformatics 26, 841-842, doi:10.1093/bioinformatics/btq033 (2010).
69 Rainer, J., Gatto, L. & Weichenberger, C. X. ensembldb: an R package to create and use Ensembl-based annotation resources. Bioinformatics 35, 3151-3153, doi:10.1093/bioinformatics/btz031 (2019).
70 Jersie-Christensen, R. R., Sultan, A. & Olsen, J. V. Simple and Reproducible Sample Preparation for Single-Shot Phosphoproteomics with High Sensitivity. Methods Mol Biol 1355, 251-260, doi:10.1007/978-1-4939-3049-4_17 (2016).
71 Tyanova, S. et al. The Perseus computational platform for comprehensive analysis of (prote)omics data. Nat Methods 13, 731-740, doi:10.1038/nmeth.3901 (2016).
72 Ameziane, N. et al. A novel Fanconi anaemia subtype associated with a dominant-negative mutation in RAD51. Nat Commun 6, 8829, doi:10.1038/ncomms9829 (2015).
73 Zerbino, D. R. et al. Ensembl 2018. Nucleic Acids Res 46, D754-D761, doi:10.1093/nar/gkx1098 (2018).
74 Cox, J. & Mann, M. MaxQuant enables high peptide identification rates, individualized p.p.b.-range mass accuracies and proteome-wide protein quantification. Nat Biotechnol 26, 1367-1372, doi:10.1038/nbt.1511 (2008).
75 UniProt, C. UniProt: a worldwide hub of protein knowledge. Nucleic Acids Res 47, D506-D515, doi:10.1093/nar/gky1049 (2019).
76 Zhang, X. et al. Proteome-wide identification of ubiquitin interactions using UbIA-MS. Nat Protoc 13, 530-550, doi:10.1038/nprot.2017.147 (2018).
77 Kalaora, S. et al. Use of HLA peptidomics and whole exome sequencing to identify human immunogenic neo-antigens. Oncotarget 7, 5110-5117, doi:10.18632/oncotarget.6960 (2016).
78 Fu, L., Niu, B., Zhu, Z., Wu, S. & Li, W. CD-HIT: accelerated for clustering the next-generation sequencing data. Bioinformatics 28, 3150-3152, doi:10.1093/bioinformatics/bts565 (2012).
79 Jurtz, V. et al. NetMHCpan-4.0: Improved Peptide-MHC Class I Interaction Predictions Integrating Eluted Ligand and Peptide Binding Affinity Data. J Immunol 199, 3360-3368, doi:10.4049/jimmunol.1700893 (2017).
80 Van der Auwera, G. A. et al. From FastQ data to high confidence variant calls: the Genome Analysis Toolkit best practices pipeline. Curr Protoc Bioinformatics 43, 11 10 11-11 10 33, doi:10.1002/0471250953.bill10s43 (2013).
81 Li, H. D., Funk, C. C. & Price, N. D. iREAD: a tool for intron retention detection from RNA-seq data. BMC Genomics 21, 128, doi:10.1186/s12864-020-6541-0 (2020).
82 Andreatta, M., Alvarez, B. & Nielsen, M. GibbsCluster: unsupervised clustering and alignment of peptide sequences. Nucleic Acids Res 45, W458-W463, doi:10.1093/nar/gkx248 (2017).
83 Vita, R. et al. The Immune Epitope Database (IEDB): 2018 update. Nucleic Acids Res 47, D339-D343, doi:10.1093/nar/gky1006 (2019).
84 Krokhin, O. V. & Spicer, V. Peptide retention standards and hydrophobicity indexes in reversed-phase high-performance liquid chromatography of peptides. Anal Chem 81, 9522-9530, doi:10.1021/ac9016693 (2009).
85 Gatto, L. & Lilley, K. S. MSnbase-an R/Bioconductor package for isobaric tagged mass spectrometry data visualization, processing and quantitation. Bioinformatics 28, 288-289, doi:10.1093/bioinformatics/btr645 (2012).
86 MacLean, B. et al. Skyline: an open source document editor for creating and analyzing targeted proteomics experiments. Bioinformatics 26, 966-968, doi:10.1093/bioinformatics/btq054 (2010).
87 Meszaros, B., Erdos, G. & Dosztanyi, Z. IUPred2A: context-dependent prediction of protein disorder as a function of redox state and protein binding. Nucleic Acids Res 46, W329-W337, doi:10.1093/nar/gky384 (2018).
88 Ingolia, N. T., Brar, G. A., Rouskin, S., McGeachy, A. M. & Weissman, J. S. The ribosome profiling strategy for monitoring translation in vivo by deep sequencing of ribosome-protected mRNA fragments. Nat Protoc 7, 1534-1550, doi:10.1038/nprot.2012.086 (2012).
89 Toebes, M. et al. Design and use of conditional MHC class I ligands. Nat Med 12, 246-251, doi:10.1038/nm1360 (2006).
90 Hadrup, S. R. et al. Parallel detection of antigen-specific T-cell responses by multidimensional encoding of MHC multimers. Nat Methods 6, 520-526, doi:10.1038/nmeth.1345 (2009).
91 Philips, D., van den Braber, M., Schumacher, T. N. & Kvistborg, P. pMHC Multiplexing Strategy to Detect High Numbers of T Cell Responses in Parallel. Methods Mol Biol 1514, 93-101, doi:10.1007/978-1-4939-6548-9_7 (2017).
92 Dong, L., Li, P., Oenema, T., McClurkan, C. L. & Koelle, D. M. Public TCR use by herpes simplex virus-2-specific human CD8 CTLs. J Immunol 184, 3063-3071, doi:10.4049/jimmuno1.0903622 (2010).
93 Perez-Riverol, Y. et al. The PRIDE database and related tools and resources in 2019: improving support for quantification data. Nucleic Acids Res 47, D442-D450, doi:10.1093/nar/gky1106 (2019).

1. A method of producing at least one out-of-frame peptide of 5-40 amino acid residues by a cell, whereby said out-of-frame peptide is caused by ribosomal bypass of a tryptophan codon in the absence of sufficient levels of tryptophan, said method comprising incubating said cell in a growth medium, reducing the amount of tryptophan in said cell, thus producing an out-of-frame peptide of 5-40 amino acid residues by the cell.

2. The method of claim 1, wherein the amount of tryptophan is reduced in said cell by providing growth medium that is depleted of tryptophan, by incubation of the cells in the presence of interferon gamma, by activation of indoleamine 2,3-dioxygenase 1 (IDO1), indoleamine 2, 3-dioxygenase 2 (IDO2), and/or tryptophan 2, 3-dioxygenase (TDO) in the cells, or a combination thereof.

3. The method of claim 1, wherein 8-22 amino acid residues comprising at least part of the out-of-frame peptide are presented by MHC on the surface of said cell, preferably by MHC class I.

4. The method of claim 1, wherein the cell is a tumor cell such as a melanoma cell, or a breast cancer cell.

5. A method of identifying at least one out-of-frame peptide of 5-40 amino acid residues, said method comprising providing a cell in which the amount of tryptophan has been reduced, and identifying at least one out-of-frame peptide of 5-40 amino acid residues that is produced by said cell, preferably identifying a peptide of 8-22 amino acid residues that is presented by WIC on the surface of said cell and which peptide comprises at least part of the out-of-frame peptide.

6. An out-of-frame peptide of 5-40 amino acid residues that is produced by a cell upon reduction of tryptophan in said cell, said out-of-frame peptide preferably is selected from Table 1.

7. A T cell epitope comprising 8-22 amino acid residues, more preferred 8-13 amino acid residues, of an out-of-frame peptide of claim 6, preferably one or more peptides with SEQ ID NOs 46-63.

8. A polyepitope, comprising 2-50, preferably 5-25 individual T cell epitopes according to claim 7, preferably each contained within a sequence of 8-40 amino acid residues, which individual epitopes may be alternated by spacer sequences, preferably of 1-10 amino acid residues.

9. A B cell epitope comprising at least one out-of-frame peptide of 5-40 amino acid residues according to claim 6.

10. A nucleic acid molecule, encoding the T cell epitope of claim 7, said nucleic acid molecule preferably being a RNA molecule, or a DNA molecule, that expresses said polyepitope upon delivery to a suitable cell.

11. A T cell, comprising a T cell Receptor (TCR) that is directed against the T cell epitope of claim 7.

12. A method of inducing an immune response in an individual against at least one out-of-frame peptide of 5-40 amino acid residues that is produced by a cell, said method comprising providing said individual with the T cell epitope of claim 7.

13. A method of treating an individual suffering from a tumor such as a melanoma, comprising providing said individual with the T cell epitope of claim 7, said individual comprising a cell that expresses the at least one out-of-frame peptide of 5-40 amino acid residues.

14. The method of claim 13, comprising providing said individual with a RNA molecule, or a DNA molecule, that expresses said polyepitope upon delivery to a suitable cell, preferably a mRNA molecule, and the T cell comprising a T cell Receptor (TCR) that is directed against the T cell epitope.

15. The method of claim 12, wherein said individual is further provided with interferon gamma, an immune checkpoint inhibitor, or both, whereby said interferon gamma, optionally combined with a tryptophan-low or tryptophan-free diet, and/or immune checkpoint inhibitor may be administered prior to, simultaneously with, or following administration of said T cell epitope.

16. The method of claim 13, wherein said individual is additionally provided with an inducer of kynureninase.

17. A pharmaceutical composition, comprising the T cell epitope of claim 7, optionally, an accessory molecule such as an adjuvant, an immune checkpoint inhibitor, an immune stimulating molecule such as a chemokine and/or a cytokine, an inducer of kynureninase, or a combination thereof.
